SEQUENCE LISTING (36 sequences)

SEQ ID NO: 1 - protein, 8 aa, Artificial Sequence
  Synthetic construct
FTATRPGM

SEQ ID NO: 2 - protein, 9 aa, Artificial Sequence
  Synthetic construct
FTATRPGMY

SEQ ID NO: 3 - protein, 9 aa, Artificial Sequence
  Synthetic construct
MYYGQCSEI

SEQ ID NO: 4 - protein, 11 aa, Artificial Sequence
  Synthetic construct
TFTATRPGMYY

SEQ ID NO: 5 - protein, 10 aa, Artificial Sequence
  Synthetic construct
TFTATRPGMY

SEQ ID NO: 6 - protein, 11 aa, Artificial Sequence
  Synthetic construct
FTATRPGMYYG

SEQ ID NO: 7 - protein, 9 aa, Artificial Sequence
  Synthetic construct
TATRPGMYY

SEQ ID NO: 8 - protein, 29 aa, Artificial Sequence
  Synthetic construct
PKIRTEQISPFFPLVSAHLSSAMTHITEG

SEQ ID NO: 9 - protein, 23 aa, Artificial Sequence
  Syntheic construct
EPKKSTSVYPNFSTTNLIGPDPT

SEQ ID NO: 10 - protein, 19 aa, Artificial Sequence
  Synthetic construct
RHGQQNFPESFDYGMSILY

SEQ ID NO: 11 - protein, 10 aa, Artificial Sequence
  Synthetic construct
DFDGTRARFL

SEQ ID NO: 12 - protein, 10 aa, Artificial Sequence
  Synthetic construct
SIDDKVGRHF

SEQ ID NO: 13 - protein, 10 aa, Artificial Sequence
  Synthetic construct
NPLDGQAEVF

SEQ ID NO: 14 - protein, 10 aa, Artificial Sequence
  Synthetic construct
IMELEPGLTY

SEQ ID NO: 15 - protein, 10 aa, Artificial Sequence
  Synthetic construct
TQAQPGDAKL

SEQ ID NO: 16 - protein, 10 aa, Artificial Sequence
  Synthetic construct
NPIIINPTSF

SEQ ID NO: 17 - protein, 10 aa, Artificial Sequence
  Synthetic construct
IRAEHISPFF

SEQ ID NO: 18 - protein, 10 aa, Artificial Sequence
  Synthetic construct
TPNGSQFLTL

SEQ ID NO: 19 - protein, 10 aa, Artificial Sequence
  Synthetic construct
ANDQQQIQVY

SEQ ID NO: 20 - protein, 9 aa, Artificial Sequence
  Synthetic construct
ASDLAQDKY

SEQ ID NO: 21 - protein, 9 aa, Artificial Sequence
  Synthetic construct
MYTYRPWTI

SEQ ID NO: 22 - protein, 9 aa, Artificial Sequence
  Synthetic construct
FQMETVNTL

SEQ ID NO: 23 - protein, 9 aa, Artificial Sequence
  Synthetic construct
SQAAPQSTW

SEQ ID NO: 24 - protein, 9 aa, Artificial Sequence
  Synthetic construct
FPKMSMLPL

SEQ ID NO: 25 - protein, 9 aa, Artificial Sequence
  Synthetic construct
LFDHAVAKF

SEQ ID NO: 26 - protein, 9 aa, Artificial Sequence
  Synthetic construct
RYDLAAAAL

SEQ ID NO: 27 - protein, 15 aa, Artificial Sequence
  Synthetic construct
SRNLIFLTRSVQTWR

SEQ ID NO: 28 - protein, 9 aa, Artificial Sequence
  Synthetic construct
FYHYYTVVY

SEQ ID NO: 29 - protein, 15 aa, Artificial Sequence
  Synthetic construct
LDKALASFRNAIRVN

SEQ ID NO: 30 - protein, 9 aa, Artificial Sequence
  Synthetic construct
AWSSLHAVF

SEQ ID NO: 31 - protein, 9 aa, Artificial Sequence
  Synthetic construct
VPYLSFMTY

SEQ ID NO: 32 - protein, 15 aa, Artificial Sequence
  Synthetic construct
SLRSVVPLPSLRSVE

SEQ ID NO: 33 - protein, 15 aa, Artificial Sequence
  Synthetic construct
DFYYLLMNSTPLQVL

SEQ ID NO: 34 - protein, 15 aa, Artificial Sequence
  Synthetic construct
GKKFRVMKTNPGYIA

SEQ ID NO: 35 - protein, 15 aa, Artificial Sequence
  Synthetic construct
RWGKLLMVKTIKSNV

SEQ ID NO: 36 - protein, 15 aa, Artificial Sequence
  Synthetic construct
LRSVVPLPSLRSVEP

(19) United States
(12) Patent Application Publication
(10) Pub. No.: US 2022/0265792 A1
(43) Pub. Date: Aug. 25, 2022

(54) METHODS FOR TREATING SOLID TUMORS

(71) Applicant: Breakbio Corp., New York, NY (US)

(72) Inventors: Devabhaktuni SRIKRISHNA, New York, NY (US); Roy DE SOUZA, New York, NY (US)

(21) Appl. No.: 17/631,170

(22) PCT Filed: Jul. 30, 2020
(86) PCT No.: PCT/US2020/044327

§ 371 Date: Jan. 28, 2022

Related U.S. Application Data
(60) Provisional application No. 62/880,614, filed on Jul. 30, 2019.
(60) Provisional application No. 62/881,887, filed on Aug. 1, 2019.
(60) Provisional application No. 62/886,946, filed on Aug. 14, 2019.
(60) Provisional application No. 62/943,155, filed on Dec. 3, 2019.

(51) Int. Cl. A61K 39/00; A61K 39/395; A61K 31/44; A61K 31/519; A61P 35/00; A61P 37/02

(52) U.S. Cl. CPC A61K 39/0011; A61K 39/3955; A61K 31/44; A61K 31/519; A61P 35/00; A61P 37/02; A61K 2039/507; A61K 2039/55516; A61K 2039/55566; A61K 2039/55583; A61K 2039/82

(57) ABSTRACT

Solid cancerous tumors are treated by administration of an antigen presenting cell agent, a T-cell activating neoantigen vaccine, and an immunosuppression inhibitor. Another aspect of the invention is a method for treating solid cancerous tumors (SCT) in a subject, by administering an antigen presenting cell agent; a T-cell activating vaccine; and an immunosuppression inhibitor.

### CROSS-REFERENCE TO RELATED APPLICATIONS

This application claims priority to U.S. Provisional Patent Application No. 62/880,614, filed Jul. 30, 2019, U.S. Provisional Patent Application No. 62/881,887, filed Aug. 1, 2019, U.S. Provisional Patent Application No. 62/886,946, filed Aug. 14, 2019, and U.S. Provisional Patent Application No. 62/943,155, filed Dec. 3, 2019, the disclosures of each of which are incorporated by reference herein in their entireties.

### INCORPORATION BY REFERENCE

This application contains a Sequence Listing which has been filed electronically in ASCII format and is herein incorporated by reference in its entirety. Said ASCII copy, created on Jul. 30, 2020, is named 056705_502001 WO_SequenceListing_07302020 and is 8,221 bytes in size.

### FIELD OF THE INVENTION

The invention relates to systems and methods for treating solid tumors.

### BACKGROUND OF THE INVENTION

Cancers with solid tumors are the most common cancers in the United States (D. Wang et al., Cancer J (2013) 19(6):502-10). Current treatments for solid tumors include surgery, chemotherapy, radiation therapy, and immunotherapy. Surgical interventions can be effective if these tumors are detected at an early stage, however, in many patients cancer is not detected until it is already at an advanced stage.

For example, colorectal cancer (CRC) is a complex disease, and other classification systems have been proposed to better describe different aspects and phases. One such system is the Immunoscore, which classifies tumors according to the presence of CD3+ and CD8+ cells (T-cells) at the tumor center and the invasive margin. Scores range from I0 (having few or no CD3+ or CD8+ cells at both the center and the margin) to I4 (having high immune cell densities in both locations). The consensus Immunoscore has been validated globally in colon cancer, and has a greater relative prognostic value than other measures such as lymphovascular invasion, tumor differentiation and microsatellite instability (MSI) status. CRC tumors are often referred to as either “hot” (I4, showing a vigorous cytotoxic lymphocyte (CTL) response) or “cold” (I0, showing little or no response). Tumors of as either “hot” (I4, showing a vigorous cytotoxic lymphocyte (CTL) response) or “cold” (I0, showing little or no response). Tumors of intermediate status can be classified as “excluded” and “immunosuppressed.” In “excluded” tumors, CTLs and dendritic cells (APCs) may be found at the margin of the tumor, but are prevented from entering. In “immunosuppressed” tumors, CTLs and APCs are found within the tumor, but are ineffective and deactivated. See, e.g., J. Galon et al., Nat Rev Drug Discov (2019) 18:197-218.

Tumors can form a dense mass, containing malignant cells, tumor-associated macrophages (TAM), tumor-associated or cancer-associated fibroblasts (TAF or CAF), extracellular matrix, and collagen. As the tumor grows, it can become hypoxic and often has a pH lower than normal tissue. Tumor cells, and other cells within the tumor, can express proteins inappropriately, such as the overexpression of CD73, which leads to immunosuppressive concentrations of adenosine, and PD-L1, which can lead to T-cell anergy. The under-expression of other proteins, such as interferon receptors and MHC-I, leads to immunity from CTL activity, as in “immunosuppressed” tumors. The tumor margin can become impassable to CTLs, as in “excluded” tumors. See, e.g., J. A. Joyce et al., Science (2015) 348(6230):74-80; R. Levayer, Seminars Cancer Biol (2019) https://doi.org/10.1016/j.semcancer.2019.05.004. The tumor milieu can cause CD8+ T-cell differentiation into suppressor/regulatory phenotypes, and induce macrophages to shift to the M2 immunosuppressive phenotype. As a result, solid tumors like CRC are more difficult to treat than many other cancers. Further, CRC encompasses at least three subtypes with unique immunological characteristics and varied immune escape mechanisms (J. Guinney et al., Nat Med (2015) 21(11):1350-56; M. A. Komor et al., J Pathol (2018) 246(3):266-276).

There are more than 2,000 immuno-oncology agents currently being tested or in use, addressing more than 60 different targets (J. Galon et al., supra). These agents, taken singly, provide a complete response in only a small minority of cases. There are numerous clinical trials in progress testing the efficacy of specific pairs of such agents: considering just the distinct targets, there are more than 60², or 3,600+ possible pairs that can be tested. To date, pairs of immune-oncology agents improve the response rate, but still ultimately fail in the majority of cases. The results of trials showing improvement when combining, for example, a CTLA-4 inhibitor (such as ipilimumab) and a PD-1 inhibitor (such a nivolumab), suggest that addressing only one immune checkpoint is not sufficient, and that most or all significant checkpoints must be addressed for full response. Treating the single category of immune checkpoint blockade alone is unlikely to be fully effective, as there are other routes of tumor escape, so it is most likely that several or all categories of targets must be addressed. Combinations of agents addressing three different targets amount to 60³=216,000 possible combinations. Four different targets: 12.9 million combinations. Five different targets: 777.6 million combinations. These figures do not include the 100+ chemotherapeutic agents that could be employed in combination, nor consideration of other agents such as COX-2 inhibitors (e.g., aspirin) or angiotensin-II receptor type 1 inhibitors (e.g., losartan) that could also be combined, nor oncogenic pathways yet to be discovered. Clearly, the global economy cannot support a systematic investigation of all possible immuno-oncology agent combinations.

The selection problem is normally addressed by means of animal models. Most basic cancer research is performed using mouse models of cancer development. In some cases, tumors are induced in mice directly, using chemical agents. These provide tumors that superficially resemble human tumors, but are not necessarily related in any other way. In other cases, human tumor cells (often from a single, standardized cell line) are implanted in mice, and the activities of candidate drugs are tested. However, the human immune system (and cancerous tissue in humans) differs from the murine system, rendering these models less predictive. This lack of prediction is evidenced by the number of drugs that show promise in mouse (and other animal) models, but fail when they reach clinical trials. This is also evidenced by unforeseen consequences such as the severe cytokine release syndrome precipitated in the first human trials of TGN1412, an anti-CD28 monoclonal antibody, which resulted in fatalities despite being administered at an initial dose 0.2% of the dose believed safe in non-human primates (see, e.g., E. W. St. Clair, J Clin Invest (2008) 118(4): 1344-47). Drug combinations predicted to work in humans based on successful animal models have failed to show repeatable efficacy in humans for some cancers, such as cobimetinib and atezolizumab for metastatic colorectal cancer (P. J. R. Ebert et al., Immunity (2016) 44(3):609-21; C. Eng et al., Lancet Oncol (2019) 20(6):849-61, https://doi.org/10.1016/51470-2045(19)30027-0), and have not proven to be a reliable way to model the human tumor microenvironment and predict the efficacy or safety of immunotherapies. For example, mouse models commonly used to test drugs for mCRC, such as CT26, use tumor cells that are perhaps representative of a small subset of patients, and would therefore provide insight regarding the response only of those patients' tumor cells to increased immune activation (J. C. Castle et al., BMC Genomics (2014) 15(1):190-201). In CRC, it is likely that different patients have different mutations, and therefore cancer cells from one patient are not necessarily representative of cancer cells in all patients. Further still, when tumor cells are tested in a mouse model (typically without adding stromal cells), this does not necessarily recreate the immunosuppression that the tumor cells had created in the human patient, for example the immunosuppression caused by the interplay between stromal cells and tumor cells. Thus, there is a need for a better way to predict the efficacy of a combination immunotherapy.

The present disclosure provide correlation of gene expression (by measurement of RNA) in patient tumors potentially offering a much better way to predict the efficacy of a combination immunotherapy across many patients. The use of gene expression data involves correlating genetic biomarkers in the patients' tissues simultaneously for multiple druggable targets of the immunotherapy combination. This also involves correlating the combined expression of one, two, or more genes in patient tumor tissues to identify which combinations of gene expressions causes immune suppression, and what drug combination may counter this cause of immune suppression.

### BRIEF DESCRIPTION OF THE DRAWINGS

FIG. 1 shows two radiologic scans of the subject described in Example 4 below. The right panel indicates the subject's condition on Sep. 9, 2019, while the left panel shows the progress of treatment on Nov. 26, 2019.

### BRIEF SUMMARY OF THE INVENTION

In general, the present disclosure determined the shortcomings of prior therapies, identified the target categories that must be addressed, and have invented an effective system and method for treating solid tumors based on relevant human data. The results herein are in part based upon data generated by the TCGA Research Network, https://www.cancer.gov/tcga.

An aspect of the invention is, inter alia, a system for treating solid cancerous tumors (colorectal, pancreatic, prostate, head and neck, melanoma, lung, liver, stomach, and breast) in a subject, the system having: an antigen presenting cell agent; a T-cell activating vaccine; and an immunosuppression inhibitor.

Another aspect of the invention is a method for treating solid cancerous tumors (SCT) in a subject, by administering an antigen presenting cell agent; a T-cell activating vaccine; and an immunosuppression inhibitor.

Another aspect of the invention is a T-cell activating vaccine having a plurality of neoantigens, or one or more nucleic acids encoding a plurality of neoantigens; and a pharmaceutically acceptable carrier.

Another aspect of the invention is a method for aiding a subject's immune response against a neoplastic disease, by administering an antigen presenting cell agent; a T-cell activating vaccine; and an immunosuppression inhibitor.

### DETAILED DESCRIPTION OF THE INVENTION

A. Systems

An aspect of the invention is the system that includes at least the following elements: an antigen presenting cell agent, a T-cell activating vaccine; and an immunosuppression inhibitor. An embodiment is the system wherein the antigen presenting cell agent is a CD40 agonist, a Toll-like receptor agonist, an adjuvant, FLT3L, or any combination thereof. An embodiment of the invention is the system wherein the immunosuppression inhibitor is a CD73 inhibitor, a PD-L1 inhibitor, a PD-1 inhibitor, an A2a receptor inhibitor, a multi-kinase inhibitor, cyclophosphamide, a COX-2 inhibitor, a prostaglandin-E2 inhibitor, or a combination thereof. An embodiment is the system that further includes an angiotensin II type 1 receptor antagonist.

1. Antigen Presenting Cell Agents

Most types of cell can present an antigen to immune cells. However, “professional” antigen presenting cells (APCs) are a rare and heterogeneous cell population with distinctive morphology and widespread tissue distribution. See, e.g., R. M. Steinman, Annu Rev Immunol (1991) 9:271-96. APCs include dendritic cells (DCs), macrophages, B-cells. DCs display an unusual cell surface phenotype, characterized by expression of the cell surface markers CD1, CD86, CD11c, DEC-205, CD40, MHC-II, and the absence of CD14 and other lineage markers. APCs are capable of sensitizing MHC-restricted T-cells, and provide an effective pathway for presenting antigens to T-cells in situ, both self antigens during T-cell development and foreign antigens during immune response.

Prior to encountering a foreign antigen, APCs express very low levels of MHC-II and co-stimulatory molecules. APCs continually sample the surrounding tissue and environment, and endocytose and process the antigens encountered. When an APC pattern-recognition receptor (see Toll-like receptors, below) recognizes a pathogen-associated molecular pattern or a damage-associated molecular pattern, the APC phagocytoses the antigen and becomes activated, upregulating the expression of MHC-II molecules and co-stimulatory molecules required for T cell activation such as CD40 and B7. The APC is then fully mature, and moves from the tissue to lymph nodes, where it encounters and activates T cells.

a. FLT3L

FMS-like tyrosine kinase 3 ligand (FLT3L, also known as FLT3LG) can be used to stimulate the generation of downstream or intermediate cells such as myeloid precursor cells, monocytic cells, macrophages, B cells, and dendritic cells from CD34+ bone marrow progenitors and stem cells. It can also be used for mobilizing antigen presenting cells in vivo, expanding antigen presenting cells ex vivo, e.g., for activation ex vivo with selected antigens and reintroduction into a subject. FLT3L and derivative polypeptides are described in U.S. Pat. No. 5,554,512, WO 94/28391, and US 20060292166, all incorporated herein by reference.

FLT3L and its derivatives are made and administered by the methods described in U.S. Pat. No. 5,554,512, WO 94/28391, and US 20060292166, and may further be administered as a nucleic acid encoding the proteins and polypeptides described therein. An embodiment is the system comprising FLT3L or a derivative thereof. Another embodiment is the system comprising the FDA approved white blood cell growth factor pegfilgrastim (Neulasta, Amgen) (https://clinicaltrials.gov/ct2/show/NCT03789097), which like FLT3L has been shown to create new dendritic cells in patients that could assist an immunogenic response (Bonanno et al, J Transl Med. 2010; 8:114)

b. Toll-Like Receptor Agonists

Toll-like receptor (TLR) agonists activate the TLR, and the thus activate the APC expressing the TLR. TLR3 can be activated by poly(I:C) and derivatives thereof (e.g. AmpliGen®, Hiltonol®, poly-ICLC, poly(IC-R), poly(I:C₁₂U), and non-CpG bacterial DNA and RNA. Poly(I:C) is a dimer of polyinosinic acid and polycytidylic acid. The double-stranded RNA structure stimulates TLR3. Poly-ICLC (Hiltonol®) is poly-(I:C) and poly-L-lysine and carboxymethyl cellulose. TLR agonists are generally administered by intradermal or intramuscular injection, and may be combined with specific antigens. One embodiment of the invention is the system comprising a TLR agonist. An embodiment of the invention is the system wherein the agonist comprises poly(I:C) or a derivative thereof. Another embodiment of the invention is the system wherein the agonist comprises poly-ICLC.

c. CD40 Agonists

CD40 is a costimulatory protein found on APCs, and is required for their activation. CD40L (also known as CD154) expression on CD4+ T-cells, and its binding to CD40, activates APCs and induces or “licenses” antigen presenting cells to mature and thereby trigger T-cell activation and differentiation. CD40 agonists (including CD40L mimics and fragments) can be used to trigger APC maturation and migration, resulting in amplification of the APC population, including APCs in and around the tumor. CD40 agonists include CD40L (whether membrane-bound or soluble), CD40 agonists (e.g., as described in US 2019071509 and U.S. Pat. No. 7,338,660, both incorporated herein by reference), anti-CD40 antibodies such as lucatumumab and dacetuzumab, and CD40 agonist peptides (for example, as described in U.S. Pat. No. 9,161,976, incorporated herein by reference).

CD40 agonists can be administered by known methods, as appropriate for the form of the agonist. Protein-based CD40 agonists can also be administered in the form of nucleic acids that encode the agonist, for example in the form of a viral vector or gene therapy vehicle. Another embodiment is the system comprising the APC cell maturation agent Maravairoc (https://www.ncbi.nlm.nih.gov/pmc/articles/PMC6685512/).

d. Adjuvants

Other compounds that are generally useful in modulating an immune response include adjuvants, for example, MONTANIDE™, squalene, muramyl di- and tri-peptides, saponins, and the like, which may be used in combination with each other, and with the other APC agents set forth above. MONTANIDE™ is an oil-in-water emulsion using a mannide-mono-oleate derivative as an emulsifier, which has been developed as a vaccine adjuvant. MONTANIDE™ can contain mineral oil, squalene, or other oils. Most adjuvants have an oil or emulsion base, and are combined with vaccine antigens prior to administration.

e. Combinations

Additionally, the APC agents set forth above can be combined, co-administered, or otherwise used together in the methods of the invention. One embodiment of the invention is the system that comprises FLT3L or a derivative thereof, and poly(I:C) or a derivative thereof. Another embodiment of the invention is the system comprising FLT3L and a CD40 agonist. Another embodiment of the invention is the system comprising FLT3L, a CD40 agonist, and poly(I:C) or a derivative thereof. Another embodiment of the invention is the system comprising a CD40 agonist and poly(I:C) or a derivative thereof. An embodiment is the system including FLT3L and poly-ICLC. An embodiment is the system including FLT3L, poly-ICLC, and MONTANIDE™.

2. T-Cell Activating Vaccines

Overall survival and progression-free survival are associated with the presence of tumor-infiltrating lymphocytes (TILs), particularly active effector T-cells (CTLs) within the tumor (J. Galon et al., Science (2006) 313:1960-64). Obtaining such CTLs requires exposure to appropriate tumor antigens, and activation by an APC. SBRT and related ablative therapies also rely on T-cell activity for abscopal effects (tumor killing outside the regions directly exposed to radiation).

a. Neoantigens and Neoepitopes

Early evidence shows that neoantigen-based vaccination or neoantigen vaccine can elicit T-cell responses, and that neoantigen targeted T-cell therapy can cause tumor regression under certain circumstances in selected patients. In order to recognize tumor cells, T-cells must bind tumor-specific antigens with high affinity, and the antigens must be sufficiently distinct from normal proteins to avoid tolerance. As tumor cells accumulate numerous mutations during their evolution into malignancy, some mutations result in alterations in the amino acid sequences of proteins expressed in the tumor. Alterations that are distinct may also be immunogenic. Antigens that are expressed by tumor cells, but not normal cells, are termed neoantigens.

Tumor cells are characteristically poorly regulated and heterogeneous, so it is likely that not all cells within a tumor will express the same set of neoantigens. Further, treatment targeting a single antigen can pressure the tumor cells to down-regulate expression of that antigen, and even down-regulate expression of MHC proteins so that few or no antigens are presented. In the absence of a neoantigen presented on an MHC-I protein on the surface of the tumor cell, a CTL is unable to recognize and kill the tumor cell. As a result, multiple neoantigens are required in order to kill as many tumor cells as possible.

Candidate neoantigens can be identified by immunological tests, sequencing (for example, deep RNA sequencing of biopsy tissue), and prediction, or a combination thereof. By sequencing biopsied or resected tumor tissue obtained from a subject, one can determine which proteins are mutated, and therefore candidate “personalized” neoantigens for that subject. Using sequencing of such tissue obtained from many subjects, one can determine which proteins are most frequently mutated, and therefore may be combined as general neoantigens formulations. As a further refinement, one may correlate neoantigen binding to specific MHC-I and MHC-II alleles, to determine or predict which neoantigens are likely to work well for a particular subject, based on that subject's personal alleles. See, e.g., M. Rajasagi et al., Blood (2014) 124(3):453-62; Y. Chu et al., Theranostics (2018) 8(15):4238-46; R. E. Hollingsworth et al., npj Vaccines (2019) 4:7-17; and P. A. Ott et al., Nature (2017) 547(7662):217-21. For exemplary techniques, see Carreno et al., WO2016/040900; Rooney, WO2017/173321; and Yelensky et al., WO2019/050994, each of which (and its published US counterpart applications) is incorporated herein by reference.

The multiple neoantigen peptides and/or multi-antigen polypeptides can be divided into a plurality of different pools, having two or more different combinations of neoantigens. For example, the vaccine may be provided in the form of 1, 2, 3, 4, 5, 6, 7, 8, 9, or about 10 separate neoantigen combinations, which combinations may or may not be unique, and which are administered to separate injection sites on the patient. The injection sites can be selected to target different lymph nodes, i.e., so that the lymph nodes that ultimately receive the neoantigen combinations are different. The targeted lymph nodes can be selected on the basis of being draining lymph nodes for one or more tumor sites.

The destruction of tumor cells bearing a particular antigen creates a selection pressure that favors the surviving cells that do not express that antigen. Thus, the best neoantigen(s) for treating a particular tumor may evolve over time as a response to treatment. See, e.g., G. Rospo et al., Genome Med (2019) 11:42-64, https://doi.org/10.1186/s13073-019-0654-6. To combat this, the system of the invention includes administration of multiple neoantigens, reanalysis of the neoantigens present in the tumor(s) as treatment progresses, and administration of updated neoantigens.

In an embodiment, the vaccine contains about 1, 2, 3, 4, 5, 6, 7, 8, 9, 10, 12, 15, 20, 25, 30, 35, 40, 45, or 50 neoantigens, which can be provided as individual peptides or concatenated into several longer multi-antigen polypeptides having about 12, 15, 20, 25, 30, 35, 40, 45, or 50 amino acids per multi-antigen polypeptide. The neoantigens and multi-antigen polypeptides can be synthesized and stored by standard methods, for example by suspension in a buffered solution and lyophilization.

The vaccine or vaccine subset combinations are formulated for administration by subcutaneous or intradermal injection. In general, such formulations will contain the neoantigens or neoantigen polypeptides in an aqueous vehicle, which can further contain buffering and suspending agents such as saline, phosphate buffered saline, surfactants, and the like. Vaccines are often formulated in oil-based or emulsion-based adjuvant compositions. In an embodiment, the neoantigen peptides or polypeptides are formulated in an oil-in-water or water-in-oil emulsion. In an embodiment of the invention, the neoantigen or multi-antigen polypeptide formulation further contains an APC agent. In an embodiment, the APC agent is FLT3L, poly-I:C, poly-ICLC, or MONTANIDE™. In the event that a neoantigen peptide or multi-antigen polypeptide is not sufficiently soluble in aqueous solution, it can be formulated directly into the oil phase of an oil-in-water or water-in-oil emulsion. In an embodiment, the neoantigen peptides and/or multi-antigen polypeptides are formulated directly into an adjuvant solution, such as MONTANIDE™.

3. Immunosuppression Inhibitors

The immune system includes regulatory mechanisms that modulate or suppress the immune response, including immune checkpoints, adenosine receptor A2AR regulation, CTLA-4, and cytokine factors such as TGF-β. These endogenous mechanisms are often used by tumor cells to evade cytotoxic T-cell responses.

a. CD73 and Adenosine

The adenosine 2a receptor (A2aR) is a G protein-coupled receptor expressed primarily on cells of hematopoietic origin, particularly activated CTLs and CD4+T_{H} cells. Activation of the receptor leads to T-cell anergy, inhibition of CTLs, and differentiation of CD8+ CTLs to immunosuppressive T_{reg} cells. Stimulation of A2aR during antigen presentation leads to immune tolerance. (P. E. Zerk et al., Blood (2008) 111:251-59.) Extracellular adenosine is generated by CD73+ cells, which is found in many types of cancer, including glioblastoma, breast cancer, CRC, ovarian cancer, gastric cancer, and gallbladder cancer. (Z-W. Gao et al., BioMed Rsch. Intl. (2014) 2014:460654.) High expression levels of CD73 (also known as ecto-5′-nucleotidase) are correlated with poor prognosis in CRC and gastric cancer. Expression of CD73 may be driven by hypoxic conditions, often found in advanced tumors. Accordingly, antagonists of A2aR, CD73, or both, can reduce or prevent T-cell anergy.

A2aR inhibitors include anti-A2aR antibodies and derivatives, as well as small molecule inhibitors such as CPI-444, PBF-509, MK-3814, and AZD4635, which are currently in clinical trials. CD73 antagonists include anti-CD73 antibodies and derivatives thereof, such as oleclumab and BMS-986179, both currently in clinical trials. Extracellular adenosine can also be reduced by administering Adagen® (PEGylated adenosine deaminase), which is currently prescribed for the treatment of severe combined immune deficiency due to adenosine deaminase deficiency. Adenosine activity at A2aR can also be antagonized or blocked by caffeine, which competes for binding to A2aR without activating it. An embodiment of the invention is a system that includes an A2aR inhibitor, a CD73 antagonist, or a combination thereof. An embodiment is the system including a CD73 antagonist and caffeine.

b. Multikinase Inhibitors

Regorafenib is a small molecule drug that inhibits multiple kinases, and targets angiogenic, stromal, and oncogenic receptor tyrosine kinases. The oncolytic activity of the compound was originally ascribed to inhibition of raf kinase and VEGFR2, but has subsequently been shown to inhibit CSF1R, TIE2, VEGFR1, VEGFR3, PDGFR-β, FGFR, KIT, RET, and BRAF. See, e.g., S. M. Wilhelm et al., Int J Cancer (2011) 129:245-55. It further inhibits soluble epoxide hydrolase (sEH). Inhibition of multiple angiogenic pathways, and oncogenic enzymes, provides a broadened anti-tumor effect.

For example, the data from CRC tumors shows that CD4 (i.e., the presence of CD4+ T-cells within the tumor) is highly correlated with expression of many of the regorafenib targets within the tumor: CSF1R (92%), VEGFR1 (also known as FLT1) (58%), VEGFR2 (also known as KDR) (62%), VEGFR3 (also known as FLT4) (66%), FGFR1 (66%), PDGFR-α (62%), and PDGFR-β (68%). It is believed that these represent checkpoints are induced in reaction to T-cell engagement with the tumors just like PD-1 and PD-L1, which are also correlated with CD4. Similar correlations exist for CD8A (representing the presence of CTLs) as well.

Synthesis and use of regorafenib is described in U.S. Pat. Nos. 7,351,834 and 9,957,232, incorporated herein by reference. An embodiment of the invention is the system comprising regorafenib, sorafenib, fruquintinib, axitinib, lenvatinib, or a related compound.

c. PD-1 and PD-L1 Inhibitors

PD-1 (programmed cell death protein 1), also known as CD279, is an immune checkpoint protein expressed on the surface of activated T-cells, B-cells, and macrophages. When PD-1 binds to PD-L1 (CD274, or B7-H1) or PD-L2, the T-cell receptor is down-regulated, which reduces the proliferation of antigen-specific T-cells and leads to immune suppression. At the same time, PD-L1 binding to T_{reg} cells reduces their apoptosis, further increasing immune suppression. PD-L1 expression is stimulated by interferon-γ (IFN-γ) in T-cells, NK cells, macrophages, myeloid DCs, B-cells epithelial cells, and vascular endothelial cells. PD-L1 is highly expressed in some tumor cells, giving them the ability to induce anergy and avoid attack by CTLs. Inhibition of either or both PD-1 and PD-L1 can reduce or prevent anergy, and restore an anti-tumor immune response.

PD-1 can be inhibited or antagonized by anti-PD-1 antibodies and derivatives thereof, such as, for example, nivolumab, pembrolizumab, cemiplimab, pidilizumab, AMP-224, AMP-514, and PDR001. PD-L1 can be inhibited or antagonized by anti-PD-L1 antibodies and derivatives thereof, such as, for example, durvalumab, atezolizumab, avelumab, BMS-936559, and CK-301. Expression and function of PD-1 and PD-L1 are regulated by receptor tyrosine kinases (TRK), and can be modulated by TRK inhibitors. An embodiment of the invention is the system including a PD-1 inhibitor, a PD-L1 inhibitor, or both. An embodiment of the invention is the system including nivolumab. An embodiment of the invention is the system including nivolumab and atezolizumab. An embodiment of the invention is the system including: nivolumab, pembrolizumab, cemiplimab, or pidilizumab; and durvalumab, atezolizumab, or avelumab.

d. CTLA-4 Inhibitors

CTLA-4 (cytotoxic T-lymphocyte-associated protein 4), CD152, is another protein that functions as an immune checkpoint and down-regulates T-cell function. CTLA-4 is constitutively expressed on T_{reg} cells, but in other T-cells is expressed only after activation. CTLA-4 binds with higher affinity to CD80 (B7-1) and CD86 (B7-2) than does CD28, and can out-compete CD28 for binding, thereby inhibiting the stimulatory signal from CD28. CTLA-4 antagonism can reduce immune suppression. Suitable CTLA-4 inhibitors include anti-CTLA-4 antibodies, for example ipilimumab and tremelimumab. An embodiment is the system including ipilimumab or tremelimumab.

e. Cyclophosphamide

Cyclophosphamide, (RS)—N,N-bis(2-chloroethyl)-1,3,2-oxazaphosphinan-2-amine 2-oxide, is an alkylating agent used to suppress the immune system. It has recently been used at low doses to deplete lymphocytes in cancer patients, after finding that T_{reg} cells take longer to recover than CTLs. See, e.g., M. Scurr et al., Clin Cancer Res (2017) 23(22):6771-80; M. Scurr et al., JAMA Oncol (2017) 3(10):e172579; V. Radojcic et al., Cancer Immunol Immunother (2010) 59:137-48. Scurr et al. found that administration of 50 mg of cyclophosphamide bid for seven days, followed by seven days off, followed by another seven days of 50 mg bid, significantly depleted T_{reg} cells, and restored the immune response to mCRC in subjects. Radojcic et al. found that cyclophosphamide treatment resulted in an increased number of new DCs, which were better able to infiltrate tumors and present tumor antigens, without inducing additional T_{reg} cells. An embodiment of the invention is the system including cyclophosphamide.

f. PGE2 Inhibitors

Prostaglandin E2 (PGE2) is a naturally-occurring prostaglandin that reduces inflammation by down-regulating T-cell interactions with APCs and altering T-cell migration behavior (A. J. Wiemer et al., J Immunol (2011) 187:3663-70). PGE2 is increased in SCT, and promotes tumor growth and development, resistance to apoptosis, proliferation, invasion and metastasis, angiogenesis, and drug resistance in SCT. It also may promote fibrosis, which helps establish the dense stroma of the tumor microenvironment, creating a physical barrier to CTL entry. PGE2 is generated by the enzymes COX-2 and mPGES-2 (microsomal prostaglandin E synthase 2, encoded by PTGES2), and may stimulate the expression of more COX-2, leading to a positive feedback loop.

PGE2 activity can be inhibited by inhibitors of mPGES-2 and/or COX-2, which may take the form of specific antibodies or small molecules. Exemplary COX-2 inhibitors include non-steroidal anti-inflammatory drugs (NSAIDs) such as aspirin, ibuprofen, naproxen, fenoprofen, flurbiprofen, ketoprofen, indomethacin, teolmetin, ketorolac, diclofenac, prioxicam, tenoxicam, phenylbutazone, mefenamic acid, meclofenamic acid, celecoxib, etoricoxib, parecoxib, nimesulide, clonixin, licofelone, and related compounds. See, e.g., A. Grancher et al., Bull Cancer (2018) 105(2):171-80; L. Emilsson et al., Aliment Pharmacol Ther (2017) 45(2):193-204. An embodiment of the invention is the system including aspirin, ibuprofen, indomethacin, and/or naproxen.

g. Combinations

Because there are multiple routes for suppressing CTL activity, it is advantageous to include countermeasures for more than one suppression route. Accordingly, an aspect of the invention is the system including combinations of two or more of a PD-1 inhibitor, PD-L1 inhibitor, CTLA-4 inhibitor, CD73 inhibitor, A2aR inhibitor, multi-kinase inhibitor, COX-2 and/or PGE2 inhibitor. An embodiment of the invention is the system that includes a PD-1 inhibitor, and a PD-L1 inhibitor. An embodiment of the invention is the system that includes a PD-1 inhibitor, a multi-kinase inhibitor, and a COX-2 inhibitor. Another embodiment is the system including a PD-1 inhibitor, a CD73 inhibitor, and a COX-2 inhibitor. Another embodiment is the system including a PD-1 inhibitor, a CD73 inhibitor, a CTLA-4 inhibitor, and a COX-2 inhibitor. An embodiment of the invention is the system that includes a PD-1 inhibitor, and one or more inhibitors selected from a PD-L1 inhibitor, a CTLA-4 inhibitor, a CD73 inhibitor, an A2aR inhibitor, a multi-kinase inhibitor, a COX-2 inhibitor, and a PGE2 inhibitor. An embodiment of the invention is the system that includes a PD-1 inhibitor and a PD-L1 inhibitor, and one or more inhibitors selected from a CTLA-4 inhibitor, a CD73 inhibitor, an A2aR inhibitor, a multi-kinase inhibitor, a COX-2 inhibitor, and a PGE2 inhibitor. An embodiment of the invention is the system that includes a PD-1 inhibitor and a PD-L1 inhibitor, and one or more inhibitors selected from a CTLA-4 inhibitor, a CD73 inhibitor, an A2aR inhibitor, a multi-kinase inhibitor, a COX-2 inhibitor, and a PGE2 inhibitor. An embodiment of the invention is the system that includes a PD-1 inhibitor and a CTLA-4 inhibitor, and one or more inhibitors selected from a PD-L1 inhibitor, a CD73 inhibitor, an A2aR inhibitor, a multi-kinase inhibitor, a COX-2 inhibitor, and a PGE2 inhibitor. An embodiment of the invention is the system that includes a PD-1 inhibitor and a CD73 inhibitor, and one or more inhibitors selected from a PD-L1 inhibitor, a CTLA-4 inhibitor, an A2aR inhibitor, a multi-kinase inhibitor, a COX-2 or PGE2 inhibitor.

An embodiment of the invention is the system that includes: a PD-1 inhibitor; a CD73 inhibitor; and a COX-2 or PGE2 inhibitor. An embodiment of the invention is the system that includes: a PD-1 inhibitor; a CD73 inhibitor; a COX-2 or a PGE2 inhibitor; and one or more inhibitors selected from a PD-L1 inhibitor, a CTLA-4 inhibitor, an A2aR inhibitor, and a multi-kinase inhibitor. An embodiment of the invention is the system that includes: a PD-1 inhibitor; a multi-kinase inhibitor; and a COX-2 or a PGE2 inhibitor. An embodiment of the invention is the system that includes: a PD-1 inhibitor; a multi-kinase inhibitor; a COX-2, or a PGE2 inhibitor; and one or more inhibitors selected from a PD-L1 inhibitor, a CTLA-4 inhibitor, an A2aR inhibitor, and a CD73 inhibitor.

An embodiment of the invention is the system that includes: a multi-kinase inhibitor, a PD-1 inhibitor, and one or more inhibitors selected from a COX-2 inhibitor, a PGE2 inhibitor, a PD-L1 inhibitor, a CTLA-4 inhibitor, an A2aR inhibitor, and a CD73 inhibitor. An embodiment of the invention is the system that includes: a multi-kinase inhibitor, a PD-1 inhibitor, and a CTLA-4 inhibitor, and one or more inhibitors selected from a COX-2 inhibitor, a PGE2 inhibitor, a PD-L1 inhibitor, an A2aR inhibitor, and a CD73 inhibitor. An embodiment of the invention is the system that includes: a multi-kinase inhibitor; a PD-1 inhibitor; a CTLA-4 inhibitor; a COX-2 inhibitor or a PGE2 inhibitor; and one or more inhibitors selected from a PD-L1 inhibitor, an A2aR inhibitor, and a CD73 inhibitor.

4. Angiotensin-II Type 1 Receptor Antagonists and Stromal Factors

Antagonists of angiotensin-II type 1 receptors (AT1R), such as losartan, normalize the collagen or interstitial matrix of solid tumors, which facilitates the distribution and penetration of the tumors by CTLs and APCs. For example, losartan reduces collagen I levels or production by carcinoma-associated fibroblasts (CAFs). It further facilitates decompression of blood vessels and vascular normalization, and improves tumor perfusion and delivery of low molecular weight chemotherapeutics and oxygen, thus enhancing the therapeutic effect of cancer therapies and immunotherapies. One can also use other angiotensin inhibitors, for example, angiotensin receptor blockers (ARBs) such as candesartan and valsartan, angiotensin converting enzyme inhibitors (ACE-I) such as lisinopril and captopril. Each of these agents may be used singly or in combination in the practice of the invention. See, e.g., Y. Zhao et al., Proc Natl Acad Sci USA (2019) 116(6):2210-19; Y. Tang et al., Drug Deliv Transl Res (2019) 9(3):615-24; J. Scott-Emuakpor et al., J Exp Ther Oncol (2017) 11(2):107-15; R. Coulson et al., Oncotarget (2017) 8(12):18640-56; R. K. Jain et al., US2013-0287688. ARBs do not directly attract CTLs, but by normalizing the tumor stroma facilitate physical entry of CTLs when they are attracted to the tumor by other elements of the invention.

Many angiotensin-II receptor type 1 antagonists have been developed for treating hypertension and/or heart failure. Suitable AT1R antagonists include losartan, candesartan, valsartan, irbesartan, telmisartan, eprosartan, olmesartan, azilsartan, fimasartan, and the like. Typical dosages of these compounds when used for hypertension range from about 4 to about 800 mg for an adult human. For losartan, the typical dose range is about 50 to 100 mg, while candesartan ranges from about 4 to 32 mg, and eprosartan ranges from about 400 to 800 mg. However, other dosages can be effective in the systems and methods of the invention, and can be determined by standard methods. An aspect of the invention is the use of an ARB in combination with other elements of the system. An embodiment is the system including an angiotensin-II receptor type 1 antagonist. An embodiment is the system including losartan.

5. Stereotactic Body Radiotherapy (SBRT)

Radiotherapy (RT) is an effective tool for the treatment of tumors and other lesions. Stereotactic body radiotherapy (SBRT), combines the principles of stereotaxy (3-D target localization) with multiple cross-fired beams from a high-energy radiation source to precisely irradiate a target within a patient. This technique allows maximally aggressive dosing of the treatment target, while normal surrounding tissue receives lower, non-injurious doses of radiation. Targeted ionizing irradiation has long been known to cause direct localized cell death, but can also induce tumor regression at non-irradiated sites (the “abscopal effect”). Local radiotherapy is known to induce an immunostimulatory form of cell death (immunogenic cell death, or “ICD”), which simulates immune responses. It is currently believed that the response is caused by the release of damage associated molecular patterns (DAMPs), which trigger antigen engulfment by APCs, and presentation to the immune system. Irradiation is also known to increase expression of MHC-I on tumor cell, improving expression of tumor cell antigens. For these reasons, it is sometimes called “in situ vaccination.” However, SBRT also upregulates IFN, TNF, IL-1α, and IL-6, and increases the priming of effector CD8+ T cells, in addition to causing expression of CXCL10 which recruits CTLs into the tumor. Therefore improved antigen expression and presentation as well as enhanced functioning of CTLs provide a sound potential rationale for an immune mediated abscopal effect. (J. Y. H. Lim et al., Cancer Immunol Immunother (2014) 63(3):259-71.)

SBRT differs from prior forms of RT in that RT often delivers comparatively lower doses of radiation (e.g., 0.5 to 2 Gy) in multiple exposures (e.g., five doses in five days), whereas SBRT more frequently delivers higher doses of radiation (e.g., about 5 to 50 Gy) divided into fewer exposures (e.g., once per week for three weeks). In embodiments of the invention, SBRT is employed at doses of at least about 1 Gray (Gy), 2 Gy, 3 Gy, 4 Gy, 5 Gy, 6 Gy, 7 Gy, 8 Gy, 9 Gy, 10 Gy, 12 Gy, 15 Gy, 20 Gy, 25 Gy, 30 Gy, 40 Gy, 50 Gy, 60 Gy, or 75 Gy. The total dose is less than about 100 Gy, 90 Gy, 80 Gy, 70 Gy, 60 Gy, 50 Gy, 40 Gy, 30 Gy, 20 Gy, 15 Gy, 14 Gy, 13 Gy, 12 Gy, 11 Gy, 10 Gy, 9 Gy, 8 Gy, 7 Gy, 6 Gy, 5 Gy, 4 Gy, 3 Gy, or 2 Gy. In embodiments of the invention, the dose is divided into about 2, 3, 4, 5, 6, 7, 8, 9, or 10 fractions.

6. Interim Chemotherapy

As there is some delay between administration of the vaccine and full priming of T-cells, it can be necessary to begin a standard or conventional therapy while neoantigens are designed, synthesized, and administered. The particular therapy employed is selected to not interfere with T-cell priming and APC expansion, such as treatment with capecitabine, with or without bevacizumab. This treatment is combined with administration of a PGE2 inhibitor (e.g., aspirin or another COX-2 inhibitor), and an angiotensin-II receptor antagonist (e.g., losartan). An embodiment is the system including treatment with capecitabine, with or without bevacizumab prior to administration of neoantigens. An embodiment is the system including treatment with bevacizumab, capecitabine, and a COX-2 inhibitor prior to administration of neoantigens. An embodiment is the system including treatment with bevacizumab, capecitabine, aspirin, and losartan prior to administration of neoantigens

B. Method of Treatment

SCTs are treated effectively by employing the system of the invention. The method of the invention can also be termed as a method of aiding treatment, or a method of assisting the immune system of a subject. An embodiment is the method wherein the SCT is selected from colorectal cancer (CRC), pancreatic cancer, prostate cancer, head and neck cancer, lung cancer, melanoma, breast cancer, liver cancer, esophageal cancer, and gastric cancer. These forms of cancer share common responses, as shown by RNA expression data in response to treatment. An embodiment is the method wherein the SCT is CRC. An embodiment is the method wherein the CRC is metastatic CRC (mCRC). An embodiment is the method wherein the cancer is microsatellite stable mCRC (MSS mCRC).

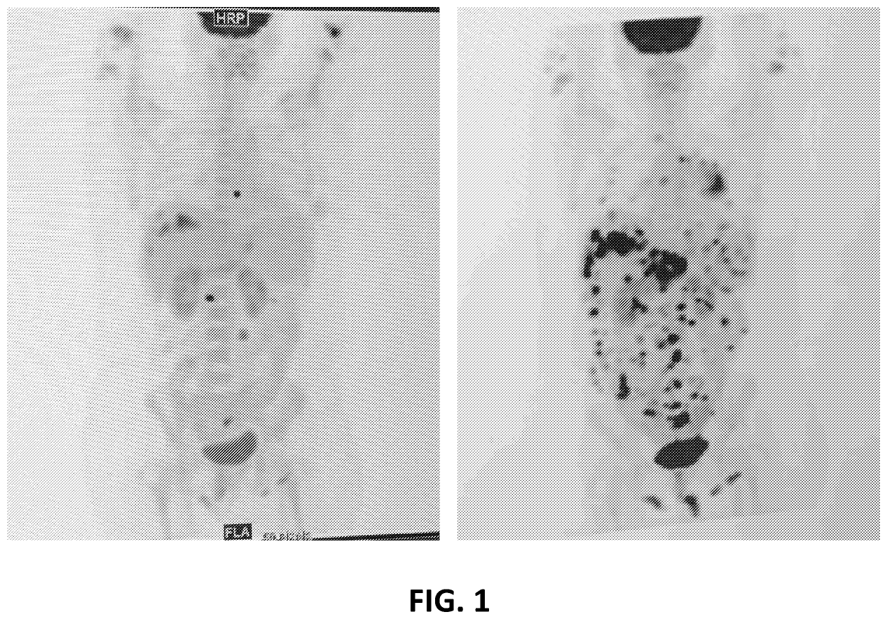

Methods of the invention include at least the following steps: administering an effective amount of an antigen presenting cell agent; administering an effective amount of a T-cell activating vaccine; and administering an effective amount of an immunosuppression inhibitor. An embodiment is the method including administering an effective amount of an antigen presenting cell agent; administering an effective amount of a T-cell activating vaccine; and administering an effective amount of an immunosuppression inhibitor. Methods of the invention can optionally include administering an angiotensin-II type 1 receptor antagonist, and/or SBRT. An embodiment of the invention is the method that further includes administering an effective amount of an angiotensin II type 1 receptor antagonist. An embodiment is the method including treatment with SBRT. An embodiment is the method including administering an angiotensin-II type 1 receptor antagonist and administering SBRT. An exemplary case of a subject receiving the presently described method of treatment is shown in FIG. 1, which shows two radiologic scans of the subject described in Example 4 below. The right panel indicates the subject's condition on Sep. 9, 2019 (highly metastasized prior to receiving the treatment described herein), while the left panel shows the progress of treatment on Nov. 26, 2019 (greatly reduced metastasis after receiving the treatment described herein).

1. Phases of Treatment

The method of the invention can be conceptually divided into three phases: antigen presenting cell activation, T-cell vaccination, and inhibition of immunosuppression. During all phases, a COX-2 inhibitor and/or an AT1R antagonist can be administered. In an embodiment, a COX-2 inhibitor is administered throughout the treatment period. In an embodiment, the COX-2 inhibitor is aspirin. In an embodiment, an AT1R antagonist is administered throughout the treatment period. In an embodiment, the AT1R antagonist is losartan. In an embodiment, both a COX-2 inhibitor and an AT1R antagonist are administered throughout the treatment period. In an embodiment, the COX-2 inhibitor is aspirin, celecoxib, ibuprofen, or naproxen, and the AT1R antagonist is losartan.

During the initial phases of treatment, prior to obtaining a specific T-cell response, standard chemotherapy is employed to prevent or delay progression of the disease before benefits from the method of the invention can be realized. A standard therapy is selected that does not interfere with antigen presenting cell or T-cell proliferation and activation. Suitable standard therapies include bevacizumab in combination with capecitabine. The standard therapy is administered upon diagnosis or shortly thereafter, and may further include administration of a COX-2 inhibitor and/or an AT1R antagonist. An embodiment of the invention is the method including treating a subject in need thereof with bevacizumab, capecitabine, a COX-2 inhibitor, and an AT1R antagonist. In an embodiment, the COX-2 inhibitor is aspirin, celecoxib, indomethacin, ibuprofen, or naproxen, and the AT1R antagonist is losartan.

Upon commencement of treatment of SCT in a patient, or shortly thereafter, samples are taken of both normal tissue and tumor tissue for purposes of sequencing. The latter may be from biopsied or resected tumor tissue. The normal tissue sample is used to determine the patient's HLA (MHC-I and -II) alleles, whether by sequencing or standard immunoassay techniques. If the patient's HLA alleles are already known, redetermination is not necessary. The tumor tissue is sequenced, such as by NGS deep sequencing, to determine neoantigens that are currently expressed in the tumor tissue. The neoantigen sequences are then analyzed for binding affinity to the patient's MHC-I and -II proteins, whether by kinetic methods (e.g., by determining the affinity constant using labeled peptides representing neoantigens competing with non-mutated “self” antigens for binding to appropriate patient cell samples) or by predictive (e.g., by computational or in silico) methods. Neoantigens that bind weakly can be improved by known methods, for example by including a strong T-cell epitope at one or both ends of the neoantigen peptide, and/or by derivatizing the neoantigen peptide to increase binding affinity to MHC-I and -II.

Neoantigen peptides are then synthesized. It is known that some mutations occur regularly in particular cancers (e.g., BRAF V600E in melanoma and CRC): for common mutations, neoantigen peptides can be synthesized and stocked in advance. See, e.g., Z. Liang et al., doi: https://doi.org/10.1101/682617 (Jul. 9, 2019). It is possible to record the incidence of each neoantigen, together with the peptide sequences found to bind to particular HLA alleles, and maintain a stock of suitable peptides for immediate use.

APCs endocytose foreign proteins and polypeptides, and process them with proteolytic enzymes that break them into oligopeptides of a size suitable for binding to MHC-I or -II. Thus, neoantigen peptides can be provided as longer multi-antigen polypeptides containing two or more neoepitopes. In an embodiment, the neoantigen peptides are administered in the form of multi-antigen polypeptides containing 1, 2, 3, 4, 5, 6, 7, 8, 9, or 10 or more neoantigens, and the multi-antigen polypeptides can contain 12, 15, 20, 25, 30, 35, 40, 45, 50, or more amino acids, or any number between those figures.

a. Antigen Presenting Cell Activation

In embodiments, FLT3L is administered to the patient in order to expand and mobilize the patient's APCs. FLT3L can be administered immediately upon commencement of treatment, one or two weeks prior to beginning T-cell activating vaccine administration, during the first week or two weeks of vaccine administration, and at the end of vaccine administration and/or one to two weeks thereafter, and combinations thereof. In an embodiment, FLT3L is administered one week prior to T-cell vaccination. In an embodiment, FLT3L is administered at the time of T-cell vaccination, or within 24 hours before or after vaccination. In an embodiment, FLT3L is administered within one week of treatment commencement, and within 24 hours of T-cell vaccination. In an embodiment, FLT3L is administered at the time of radiation therapy, or within 24 hours before or after radiation therapy.

Other APC agents are administered at or near the time of T-cell vaccination. In an embodiment, one or more additional APC agents are combined with the T-cell activating vaccine or formulated with the T-cell activating vaccine for administration. In an embodiment, an APC agent is MONTANIDE™. In an embodiment, the APC agent is MONTANIDE™ ISA51. In an embodiment, the APC agent is poly-I:C. In an embodiment, the APC agent is poly-ICLC. In an embodiment, the APC agent is MONTANIDE™ and poly-ICLC.

b. T-Cell Vaccination

As set forth herein, the T-cell activating vaccine comprises neoantigen peptides or multi-antigen polypeptides, where the neoantigens are selected based on (a) immunogenicity (which depends in part on the degree of difference between the neoantigen and the non-mutated “self” sequence peptide), (b) binding affinity to MHC-I and —II, and (c) degree of expression in the tumor tissue(s). Because tumors are characteristically heterogeneous, the vaccine contains multiple neoantigens. As described herein, the vaccine is administered as one or more subset combinations of neoantigens, at different injection sites.

The T-cell activating vaccine is administered as soon as practicable, or as soon as the APCs have been activated and had time for their population to expand. The vaccine can be administered multiple times, for example about every 1, 2, 3, 4, 5, or 6 weeks, and can contain different neoantigens and combinations of neoantigens each time, and can be administered to different injection sites. Each administration can be accompanied by administration of an APC agent, within about one week before or after vaccine administration. An embodiment is the method wherein the T-cell activating vaccine is administered within 24 hours of FLT3L administration. An embodiment is the method wherein the T-cell activating vaccine is administered between 1 day and 30 days after FLT3L administration. An embodiment is the method wherein the T-cell activating vaccine is administered between 7 days and 20 days after FLT3L administration. An embodiment is the method wherein the T-cell activating vaccine is administered in combination with an adjuvant, poly(I:C), or poly-ICLC.

c. Radiation Therapy

Radiation therapy (RT) can be used in the method of the invention, where there are tumors of sufficient size, and in positions accessible to RT without unacceptable damage to surrounding tissue. In an embodiment of the invention, stereotactic body radiation therapy (SBRT) is administered before or after completion of T-cell vaccination. In an embodiment, SBRT is administered about 1 day, 2 days, 3 days, 5 days, 1 week, or 2 weeks following the last vaccine administration. In an embodiment, SBRT is administered about 1 day, 2 days, 3 days, 5 days, 1 week, or 2 weeks before the first vaccine administration. In an embodiment, SBRT is administered between the first and last vaccine administrations. In an embodiment, SBRT is administered at an intensity of at least about 1, 2, 3, 4, 5, 6, 7, 8, 9, 10, 11, or 12 Grays (Gy). In an embodiment, SBRT is administered at an intensity of no more than about 60, 50, 40, 30, 25, 22, 20, 19, 18, 17, 16, 15, 14, 13, 12, 11, or 10 Gy. In an embodiment, SBRT is administered at an intensity of between about 5 Gy and about 10 Gy. In an embodiment, SBRT is administered in about 2, 3, 4, 5, 6, 7, 8, 9, or 10 fractions. In an embodiment, SBRT is administered in about 5 fractions. In an embodiment, FLT3L is administered within about one week of SBRT administration, before, after, or during. In an embodiment, FLT3L is administered within 24 hours of initial SBRT administration. In an embodiment, FLT3L is administered 1, 2, 3, 4, 5, 6, 7, 8, 9, or 10 times on consecutive days, by intradermal or subcutaneous injection. In an embodiment, the amount of FLT3L administered in each injection is at least about 10, 20, 30, 40, 50, 60, 70, 75, 80, 85, 90, 95, 100, 125, 150, 175, 200, 250, 300, 350, 400, 450, 500, 550, 600, 700, 750, 800, 900, or 1000 μg/Kg. In an embodiment, the amount of FLT3L administered in each injection is no more than about 5000, 4000, 3000, 2000, 1500, 1000, 900, 800, 700, 600, 550, 500, 450, 400, 350, 300, 250, or 200 μg/Kg.

d. Immunosuppression Inhibition

An immunosuppression inhibitor is administered in conjunction with vaccine administration. In an embodiment, one or more immunosuppression inhibitors is/are administered within about one week of the completion of vaccine administration, before, after, or at the time of last administration. In an embodiment, the immunosuppression inhibitor is a CD73 inhibitor. In an embodiment, the CD73 inhibitor is oleclumab (MED19447). In an embodiment, the CD73 inhibitor is BMS-986179 (Bristol-Myers Squibb), AB680 (Arcus Biosciences), CB-708 (Calithera Biosciences, Inc.), CPI-006 (Corvus Pharmaceuticals), or α,β-methyleneadenosine 5′-diphosphate sodium salt. In an embodiment, the immunosuppression inhibitor is an A2aR inhibitor. In an embodiment, the immunosuppression inhibitor is a PD-1 inhibitor. In an embodiment, the immunosuppression inhibitor is a PD-L1 inhibitor. In an embodiment, the PD-1 inhibitor is nivolumab. In an embodiment, the immunosuppression inhibitor is a CTLA-4 inhibitor. In an embodiment, the CTLA-4 inhibitor is ipilimumab.

In an embodiment, the immunosuppression inhibitor is a combination of inhibitors, and can be administered together or individually. In an embodiment, the combination includes a CD73 inhibitor and a PD-1 inhibitor. In an embodiment, the combination is oleclumab and nivolumab. In an embodiment, the combination includes a CD73 inhibitor, a CTLA-4 inhibitor, and a PD-1 or PD-L1 inhibitor. In an embodiment, the combination is nivolumab, ipilimumab, and oleclumab. An embodiment is the method including administration of a PD-1 inhibitor, and one or more inhibitors selected from a PD-L1 inhibitor, a CTLA-4 inhibitor, a CD73 inhibitor, an A2aR inhibitor, a multi-kinase inhibitor, a COX-2 inhibitor, and a PGE2 inhibitor. An embodiment is the method including administration of a PD-1 inhibitor and a PD-L1 inhibitor, and one or more inhibitors selected from a CTLA-4 inhibitor, a CD73 inhibitor, an A2aR inhibitor, a multi-kinase inhibitor, a COX-2 inhibitor, and a PGE2 inhibitor. An embodiment is the method including administration of a PD-1 inhibitor and a PD-L1 inhibitor, and one or more inhibitors selected from a CTLA-4 inhibitor, a CD73 inhibitor, an A2aR inhibitor, a multi-kinase inhibitor, a COX-2 inhibitor, and a PGE2 inhibitor. An embodiment is the method including administration of a PD-1 inhibitor and a CTLA-4 inhibitor, and one or more inhibitors selected from a PD-L1 inhibitor, a CD73 inhibitor, an A2aR inhibitor, a multi-kinase inhibitor, a COX-2 inhibitor, and a PGE2 inhibitor. An embodiment is the method including administration of a PD-1 inhibitor and a CD73 inhibitor, and one or more inhibitors selected from a PD-L1 inhibitor, a CTLA-4 inhibitor, an A2aR inhibitor, a multi-kinase inhibitor, a COX-2 or PGE2 inhibitor.

An embodiment is the method including administration of a multi-kinase inhibitor, a PD-1 inhibitor, and one or more inhibitors selected from a COX-2 inhibitor, a PGE2 inhibitor, a PD-L1 inhibitor, a CTLA-4 inhibitor, an A2aR inhibitor, and a CD73 inhibitor. An embodiment is the method including administration of a multi-kinase inhibitor, a PD-1 inhibitor, and a CTLA-4 inhibitor, and one or more inhibitors selected from a COX-2 inhibitor, a PGE2 inhibitor, a PD-L1 inhibitor, an A2aR inhibitor, and a CD73 inhibitor. An embodiment is the method including administration of a multi-kinase inhibitor; a PD-1 inhibitor; a CTLA-4 inhibitor; a COX-2 inhibitor or a PGE2 inhibitor; and one or more inhibitors selected from a PD-L1 inhibitor, an A2aR inhibitor, and a CD73 inhibitor.

The immunosuppression inhibitors are administered pursuant to their approved dosages and schedules. Administration is continued throughout the course of treatment, unless otherwise indicated.

e. AT1 Antagonists and Treatment of Stroma

The dense stromal barrier is treated with antagonists of angiotensin-II type 1 receptors (AT1R), angiotensin receptor blockers (ARBs), and/or ACE inhibitors. Even tumors that do not display an “excluded” phenotype can be treated with these agents to normalize the stroma and prevent the development of an exclusive barrier. In an embodiment of the invention, the AT1R antagonist is losartan. In an embodiment of the invention, the AT1R antagonist, ARB, or ACE inhibitor is administered beginning at or near the time of treatment commencement. In an embodiment, the AT1R antagonist, ARB, or ACE inhibitor is first administered within about one week of treatment commencement. In an embodiment, administration of the AT1R antagonist, ARB, or ACE inhibitor is continued substantially throughout the course of treatment. An embodiment is the method including administration of losartan substantially throughout the course of treatment.

2. Timing and Sequence

The timing of the phases of treatment can vary depending on patient responses. In an embodiment, the patient is treated with bevacizumab, capecitabine, losartan, and aspirin, ibuprofen, or naproxen until the T-cell activating vaccine can be administered. The vaccine is administered about 1, 2, or 3 times, at intervals of about 1 week to about 6 weeks, at intervals of about 2 weeks to about 5 weeks, at intervals of about 4 weeks, or at intervals of about one month. In an embodiment, the vaccine is administered 1, 2, or 3 times at intervals of about one month. FLT3L is administered up to about one week prior to each T-cell vaccination, or at the time of one or more T-cell vaccinations. In an embodiment, FLT3L is administered with each vaccination.

SBRT is administered before vaccination, during the vaccination phase, or after the end of the vaccination phase, if appropriate. An embodiment is the method wherein SBRT is administered about 1, 2, 3, 4, or 5 weeks after the last vaccination. An embodiment is the method wherein SBRT is administered about 1, 2, 3, 4, or 5 weeks before the first vaccination. An embodiment is the method wherein SBRT is administered between the first and the last vaccination. FLT3L can also be administered in conjunction with SBRT. In an embodiment of the invention, SBRT is administered about 1, 2, 3, 4, or 5 weeks after the last vaccination. An embodiment is the method wherein SBRT is administered about 1 week after the last vaccination. An embodiment is the method wherein FLT3L is administered at a time within about 1 week before SBRT to about 24 hours after SBRT treatment. An embodiment is the method wherein SBRT is administered at an intensity of at least about 1, 2, 3, 4, 5, 6, 7, 8, 9, 10, 11, or 12 Grays (Gy). In an embodiment, SBRT is administered at an intensity of no more than about 60, 50, 40, 30, 25, 22, 20, 19, 18, 17, 16, 15, 14, 13, 12, 11, or 10 Gy. In an embodiment, SBRT is administered at an intensity of between about 5 Gy and about 10 Gy. In an embodiment, SBRT is administered in about 2, 3, 4, 5, 6, 7, 8, 9, or 10 fractions. In an embodiment, SBRT is administered in about 5 fractions.

An immunosuppression inhibitor can be administered at any time during the method. An embodiment is the method wherein a PD-1 inhibitor and/or a PD-L1 inhibitor is administered, and a CD73 inhibitor is administered. An embodiment is the method wherein a PD-1 inhibitor and/or a PD-L1 inhibitor is administered, and multi-kinase inhibitor is administered. An embodiment is the method wherein nivolumab and regorafenib are administered. An embodiment is the method wherein administration of an immunosuppression inhibitor is initiated between the last vaccine administration and about 6 weeks after the last vaccination. An embodiment is the method wherein administration of an immunosuppression inhibitor is initiated within one week of the last vaccination.

3. Measurement of Response

The tumor response is monitored. If progression occurs, the immunosuppression inhibitor combination is changed. In an embodiment, administration of nivolumab and regorafenib is changed to nivolumab and a CD73 inhibitor. In an embodiment, administration of nivolumab and a CD73 inhibitor is changed to nivolumab and regorafenib. If progression continues, new neoantigens are designed, and the vaccination phase is repeated, followed by SBRT and administration of immunosuppression inhibitors. Measurement of Response

The patient's response to therapy can be measured in multiple ways. For example, the effects on the patient's tumor(s) can be determined by X-ray measurement of tumor shrinkage. The tumor can be biopsied or resected, and the tissue examined histologically for death of tumor cells and infiltration of CTLs and APCs. Biopsy or resection tissue can be examined by deep sequencing for changes in tumor markers, changes in heterogeneity, and the like. Blood can be examined for a decrease in circulating tumor cells, tumor DNA, and/or tumor antigens, for an increase in circulating CTLs and migrating APCs, and for a change in cytokine levels or other biomarkers. Such diagnostic results can be used to determine when to proceed to the next phase (e.g., when the APC population has sufficiently expanded, when sufficient CTLs have been produced, when immunosuppression appears or increases). Diagnostic results can also be employed to determine whether a particular agent lacks efficacy against the patient's tumor(s), and when to switch to an alternate agent. The tumor response can also be measured by measurement of biomarkers.

In an embodiment of the invention, the activation state, population size, or distribution of T-cells is determined about 5 days to about 30 days after administering the T-cell activating vaccine. In an embodiment of the invention, the tumor reaction to T-cell vaccination is determined prior to repeating the vaccination.

### EXAMPLES

The following examples are provided as a guide for those of ordinary skill in the art, and are not intended to limit the scope of the claimed invention in any way. When expression levels of a gene is provided for a tumor tissue sample, unless otherwise noted, it is understood that it refers to RNA expression of that gene (RNASeq) in the tissue sample, which is shown to produce comparable results to microarray technologies in term of expression profiling (Guo et al., PLoS One (2013) 8(8):e71462).

### Example 1: T-Cell Co-Stimulatory Proteins

The expression in tumor-infiltrating immune cells of CD4, CD3D (which forms part of the TCR-CD3 antigen receptor complex), and CD8A was compared with expression in SCT of CD80 and CD86, proteins co-stimulatory for T-cells. The results are shown in Table 1.

TABLE 1
Pearson Correlation (%) of Expression in SCT

| Solid | Number | | | | |
|---|---|---|---|---|---|
| Cancerous | of patient | | | | |
| Tumor (SCT) | tumors | CD86:CD4 | CD80:CD4 | CD86:CD3D | CD86:CD8A |
| Colorectal | 367 | 92 | 81 | 60 | 59 |
| Pancreatic | 179 | 87 | 62 | 41 | 60 |
| Prostate | 492 | 91 | 76 | 61 | 68 |
| Head and Neck | 519 | 81 | 70 | 46 | 51 |
| Melanoma | 471 | 88 | 71 | 72 | 72 |
| Lung | 969 | 83 | 62 | 51 | 48 |
| Esophageal | 182 | 64 | 61 | 42 | 42 |
| Liver | 369 | 74 | 73 | 59 | 76 |
| Stomach | 408 | 78 | 60 | 47 | 47 |
| Breast | 1085 | 86 | 63 | 49 | 58 |

These results demonstrate that expression of these characteristic T-cell antigens correlates well with expression of the T-cell activating proteins CD80 and CD86 in the SCT set forth above. This indicates that APC activating agents that increase the number of mature APCs in CRC tumors (which express CD80 and CD86) will promote T-cell infiltration, and provide a synergistic result with a T-cell activating vaccine. This is consistent with the results in which higher numbers of mature APCs in the invasive margin of CRC tumors predicts better survival in CRC (A. Pryczynicz et al., Gastroenterol Res Pract (2016) 2016:2405437).

### Example 2: Correlation of PTGES2 with CD4, CD83, and CD86

The expression of PTGES2 in CRC tumors versus expression of CD4 (a T_{H} marker), CD83 (an APC maturation marker), and CD86 (also known as B7-2, an APC marker) was examined. The results are shown in Table 2.

TABLE 2
Pearson Correlation (%) of Expression in SCT Tumors

| Solid | Number | | | |
|---|---|---|---|---|
| Cancerous | of patient | | | |
| Tumor (SCT) | tumors | PTGES2:CD4 | PTGES2:CD83 | PTGES2:CD86 |
| Colorectal | 367 | −19 | −13 | −15 |
| Pancreatic | 179 | −27 | −18 | −29 |
| Prostate | 492 | −8 | −13 | −11 |
| Head and Neck | 519 | −14 | −9 | −12 |
| Melanoma | 471 | −11 | −2 | −20 |
| Lung | 969 | −23 | −10 | −20 |
| Esophageal | 182 | −18 | −9 | −13 |
| Liver | 369 | 3 | 2 | 7 |
| Stomach | 408 | −5 | −7 | −8 |
| Breast | 1085 | −5 | −3 | −9 |

These results indicate that expression of PTGES2 (which increases production of PGE2) is associated with down-regulation of T-cells and APCs in colorectal, pancreatic, prostate, head and neck, melanoma, lung, and esophageal cancers. See also T. Seo et al., Virchows Archiv (2009) 454(6):667-76, who reported that PGE synthases are overexpressed in CRC tumors, and correlated with poorer prognosis.

The use of aspirin to inhibit COX-2—and therefore PGE2 synthesis—has been reported to improve survival in CRC patients. See, for example, Y. Cao et al., Gastroenterol (2016) 151(5):879-92, reporting reduction of risk of CRC with low TIL in patients who regularly use aspirin; and T. Hamada et al., J Clin Oncol (2017) 35(16):1836-44, reporting that aspirin use correlated positively with cancer-specific survival and overall survival in CRC patients with low PD-L1 expression, although there was no benefit in patients with high expression of PD-L1.

### Example 3: Correlation of FLT3L Expression with CD4 and CD8

The correlations of FLT3LG expression and CD4, CD8A, CD8B, and CD86 expression was compared with the correlations of CD40 expression and CD4, CD8A, CD8B, and CD86 expression in SCT. The results are shown in Table 3.

TABLE 3
Pearson Correlation (%) of Expression in SCT

| | # of patient | | | | | | |
|---|---|---|---|---|---|---|---|
| (SCT) | tumors | FLT3LG:CD4 | FLT3LG:CD8A | FLT3LG:CD86 | CD40:CD4 | CD40:CD8A | CD40:CD86 |
| Colorectal | 367 | 71 | 56 | 65 | 56 | 48 | 57 |
| Pancreatic | 179 | 35 | 42 | 19 | 49 | 44 | 42 |
| Prostate | 492 | 59 | 53 | 50 | 54 | 51 | 47 |
| Head and Neck | 519 | 49 | 61 | 40 | 44 | 42 | 44 |
| Melanoma | 471 | 67 | 50 | 59 | 61 | 48 | 62 |
| Lung | 969 | 50 | 46 | 39 | 35 | 30 | 39 |
| Esophageal | 182 | 60 | 63 | 46 | 42 | 36 | 37 |
| Liver | 369 | 45 | 55 | 51 | 30 | 34 | 37 |
| Stomach | 408 | 55 | 54 | 27 | 51 | 51 | 39 |
| Breast | 1085 | 48 | 59 | 31 | 63 | 56 | 59 |

The results demonstrate that CD4, CD8, and CD86 expression are strongly correlated with FLT3LG expression in colorectal, prostate, head and neck, melanoma, lung, esophageal, liver, stomach, breast, and kidney (renal cell carcinoma) cancers and additionally with CD40 in pancreatic cancer.

M. A. Morse et al., J Clin Oncol (2000) 18(23):3883-93 reported that administration of FLT3L (20 μg/Kg/day) to mCRC patients for 14 days (1 to 3 cycles at monthly intervals) significantly increased the number of leukocytes in peripheral blood (from 5,900/mm³ to 11,200/mm³), the percentage of APCs in PBMCs (from 2.4% to 8.8%), and the number of APCs observed on the periphery of tumors.

### Example 4: Clinical Treatment

Subject DS was diagnosed with mCRC, stage IV, and was placed on standard of care chemotherapy (FOLFOX—folinic acid, fluorouracil, and oxaliplatin), then FOLFIRI (folinic acid, fluorouracil, and irinotecan) while her neoantigens were analyzed. Fresh frozen tumor samples from the primary resection of the tumor, and normal tissue samples were transferred to Avera Institute for Human Genetics (Sioux Fall, S. Dak.) and for whole exome sequencing and HLA typing. The results were analyzed using Vaxrank and MHCflurry (OpenVax, Mt. Sinai, New York, N.Y.). See, e.g., A. Rubinsteyn et al., Front Immunol (2017) 8:1807.

The software identified a mutation in MT-CO2 (cytochrome C oxidase subunit 2), and predicted a series of peptides that were predicted to bind strongly to the subject's MHC proteins. The top candidates are shown in Table 4.

TABLE 4
Neoantigen Peptides

| | | | SEQ |
|---|---|---|---|
| | | | ID |
| Sequence | Predicted IC₅₀ | | NO: |
| FTATRPGM | 23.3 | nM | 1 |
| FTATRPGMY | 46.60 | nM | 2 |
| MYYGQCSEI | 111.30 | nM | 3 |
| TFTATRPGMYY | 352.80 | nM | 4 |
| TFTATRPGMY | 765.90 | nM | 5 |
| FTATRPGMYYG | 1076.00 | nM | 6 |
| TATRPGMYY | 1635.30 | nM | 7 |

The top two peptides (SEQ ID NOs: 1-2) were selected for use, along with 28 other peptides based on neoantigens in the following genes: NONO, TANGO6, ADAM19, HLA-DRA, DMKN, ELL, SMURF2, ARID4A, HACL1, BRIX1, NTRK2, CDC42, LPCAT3, NRAS, and NUP85. The peptides ranged in size from nine to 25 amino acids in length. The peptides were synthesized commercially, purified, and lyophilized for storage until administration.

At the time of administration, the peptides (60 μg each) were combined in six groups of five, and suspended in water for injection with 10% DMSO (1.0 mL each). The peptide mixtures were then combined with MONTANIDE™ ISA 51 (Seppic, France) in a 1:1.4 ratio. The six formulations were then administered intradermally to the upper arms. The groups of peptide formulations were administered three times, each approximately one month apart. The third administration formulations further included poly:ICLC (Hiltonol®, Oncovir, Inc., 1.8 mg/mL) at a ratio of 3:1 peptides:Hiltonol®.

Therapy included FOLFIRI with bevacizumab when the vaccine was administered. Following the third administration in January 2019, FOLFIRI was discontinued, and the subject was treated with capecitabine (2,000 mg daily one week on and one week off), bevacizumab (5 mg/kg every 2 weeks), aspirin (750 mg per day), celecoxib (a COX-2 inhibitor, 200 mg per day), and losartan (100 mg per day).

Approximately six months following the last administration of the first vaccine, a new set of 29 neoantigen peptides was designed and prepared (Table 5). Peptides were suspended in water for injection with 10% DMSO (60 μg of each peptide) in three separate formulations. The three peptide suspensions (290 μL each) were then individually combined with Hiltonol® (110 μL) and MONTANIDE™ (400 μL), and the resulting formulations were injected subcutaneously into the thighs. The subject then received two doses of CPI-006 in late July and mid-August 2019, a humanized anti-CD73 IgG1 antibody (18 mg/Kg, Corvus Pharmaceuticals) and one dose of pembrolizumab, a humanized anti PD-1 antibody (100 mg, Keytruda, Merck) in late August 2019 followed by four daily doses of Regorafenib, a multikinase inhibitor (40 mg, Bayer) in late August 2019.

TABLE 5
Neoantigen Peptides

| | SEQ |
|---|---|
| | ID |
| Sequence | NO: |
| PKIRTEQISPFFPLVSAHLSSAMTHITEG | 8 |
| EPKKSTSVYPNFSTTNLIGPDPT | 9 |
| RHGQQNFPESFDYGMSILY | 10 |
| DFDGTRARFL | 11 |
| SIDDKVGRHF | 12 |
| NPLDGQAEVF | 13 |
| IMELEPGLTY | 14 |
| TQAQPGDAKL | 15 |
| NPIIINPTSF | 16 |
| IRAEHISPFF | 17 |
| TPNGSQFLTL | 18 |
| ANDQQQIQVY | 19 |
| ASDLAQDKY | 20 |
| MYTYRPWTI | 21 |
| FQMETVNTL | 22 |
| SQAAPQSTW | 23 |
| FPKMSMLPL | 24 |
| LFDHAVAKF | 25 |
| RYDLAAAAL | 26 |
| SRNLIFLTRSVQTWR | 27 |
| FYHYYTVVY | 28 |
| LDKALASFRNAIRVN | 29 |
| AWSSLHAVF | 30 |
| VPYLSFMTY | 31 |
| SLRSVVPLPSLRSVE | 32 |
| DFYYLLMNSTPLQVL | 33 |
| GKKFRVMKTNPGYIA | 34 |
| RWGKLLMVKTIKSNV | 35 |
| LRSVVPLPSLRSVEP | 36 |

Results:

Prior to the first vaccination, the subject exhibited growing disease while on FOLFOX treatment. Following the third vaccination, the subject exhibited stable disease on Capecitabine, Bevacizumab, aspirin, losartan and celecoxib for 5 months which is not unexpected for heavily pretreated patients on Aspirin plus Capecitabine and Bevacizumab (Giampieri et al., Clinical Colorectal Cancer, Vol. 16, No. 1, 38-43). After 5 months, the subject stopped Bevacizumab to receive a CD73 inhibitor, and saw suspected progression based on slight rise in CEA and suspected lymph node metastasis visible on PET (positron emission tomography) scan. The subject was administered one dose of the new 29 peptide vaccine and two doses of CD73 inhibitor with one dose of anti PD-1, and saw dramatic progression of metastases and new lesions on PET scan on September 2019, as shown in FIG. 1 (right panel). As a result, the subject discontinued CPI-006 and resumed FOLFIRI with bevacizumab and Neulasta while maintaining aspirin, celecoxib and losartan. Two weeks after the first biweekly doses of FOLFIRI, Bevacizumab and Neulasta the CEA levels had not fallen but risen marginally to 18.6, so Maraviroc was added to FOLFIRI, bevacizumab and Neulasta with aspirin, celecoxib and losartan continuing.

Two weeks after the 2^{nd} dose of FOLFIRI, the CEA had fallen to 8.4, and the subject switched from FOLFIRI to oral Capecitabine, which is easier to tolerate and to administer. From that point onwards, the subject continued on a treatment regimen of Capecitabine (2,000 mg twice daily one week on and one week off), Bevacizumab (every two weeks), Neulasta (6 mg every two weeks), Maraviroc (150 mg twice per day), one dose of pembrolizumab (anti PD-1, 100 mg, Keytruda, Merck), Aspirin, Celecoxib, and Losartan. Approximately two and a half months later, the metastases were greatly reduced, as shown in FIG. 1 (left panel) and described below in the comparison of PET scans between Sep. 9, 2019 and Nov. 26, 2019 and confirmed by a reduction in blood marker CEA from 17.2 ng/mL to 2.9 ng/mL during the same period. The radiologist who conducted the PET CT scan on Nov. 26, 2019 stated the following Impression in the report: “Since FDG PET/CT Sep. 9, 2019, hypermetabolic malignant soft tissue metastases overall markedly diminished, with residual disease as described.”

The detailed comments of the radiologist are included below. In particular, the observation of rapid reductions in SUV (Standardized uptake value) and resolutions (“resolved”) are highly unexpected for late-stage, pretreated subjects with MSS mCRC such as DS.

“LUNGS: No suspicious hypermetabolic findings, currently. Prior scattered hypermetabolic pulmonary metastases predominantly resolved; with a few scattered residual mildly FDG-avid, diminished pulmonary nodules, for example: *Left lower lobe pulmonary nodule, CT image 94, PET image 90 (allowing for some imprecision in PET-CT fusion-localization); 0.7 cm previously 0.9 cm, SUV 2.9 previously 4.9.

PLEURA/PERICARDIUM: Prior trace effusions resolved

THORACIC NODES: CT evaluation limited by lack of IV contrast and low CT mA/dose Prior hypermetabolic mediastinal/hilar adenopathy predominantly resolved; minimal residual adenopathy; for example, *Subcarinal focus SUV 2.7 previously 6.4.

HEPATOBILIARY: Hypermetabolic capsular/subcapsular hepatic implants markedly diminished in extent scintigraphically, with significant residual viable disease; for example, *PET image 104, SUV 10.8 previously 19.6.

SPLEEN: Prior hypermetabolic perisplenic implants no longer evident scintigraphically.

PANCREAS: No abnormal uptake.

ADRENAL GLANDS: No abnormal uptake.

KIDNEYS/URETERS/BLADDER: Excreted activity is present.

ABDOMINOPELVIC NODES: Prior hypermetabolic abdominopelvic adenopathy resolved.

BOWEL/PERITONEUM/MESENTERY: *Subcutaneous ventral abdominal wall port, with attached intraabdominal/peritoneal catheter extending up into the right perihepatic region; where the catheter penetrates ventral abdominal musculature, there again visualized is focal hypermetabolic activity—possibly infectious/inflammatory versus tumor; unchanged; with SUV 22.7 previously 20.0. *Subcutaneous hepatic arterial pump reservoir with catheter in expected hepatic perihilar region; where the catheter penetrates ventral abdominal musculature, there again visualized is focal hypermetabolic activity—diminished, with SUV 4.6 previously 12.7. *hypermetabolic nodular lesion within ventral abdominal wall midline scar. Image 158, SUV 8.6 previously 22.4. Prior hypermetabolic peritoneal and other abdominal implants predominantly resolved, with residual disease; for example, *inseparable from colon anastomosis, image 203, SUV 11.7 previously 36.7. Prior small ascites resolved.

PELVIC ORGANS: No abnormal uptake.

BONES/SOFT TISSUES: No suspicious bone lesions. Diffuse hypermetabolic activity in skeletal red marrow regions, consistent with physiological variant uptake.”

### Example 5: Regorafenib Treatment

High expression of PD-L1 or PD-1, along with high expression of CD8 in CRC indicates that T-cells are actively engaging the tumor, and therefore that anti-PD-1 or anti-PD-L1 help reduce immunosuppression (A. P. R. Bally et al., J Immunol (2016) 196:2431-37; A. M. Valentini et al., Oncotarget (2018) 9:8584-96). For example, PD-1 expression on T-cells reflects avidity and anti-tumor reactivity (S. Simon et al., Oncoimmunol (2018) 7(1):e1364828). See also J. H. Park et al., J Clin Oncol (2018) 36(4 supp):631, who reported that PD-L1 expression did not correlate with clinicopathological or tumor microenvironment characteristics in patients with mismatch repair-competent CRC after resection of tumors (grades TNM I-III CRC). TIL PD-1 expression was not associated with clinicopathological characteristics, but was associated with a high Klintrup-Makinen grade (P<0.001), high Immunoscore (P<0.001), low tumor stromal percentage (TSP, P=0.068) and a low Glasgow microenvironment score (P<0.001). Multivariate survival analysis showed that high TIL PD-1 expression was associated with improved cancer-specific survival (HR 0.60, P=0.016) independent of Immunoscore (HR 0.74, P=0.03) and TSP (HR 1.91, P=0.027). PD-L1 expression was not associated with CSS on univariate or multivariate analysis.

Recent clinical trial results found that treatment the multi-kinase inhibitor regorafenib exhibited synergistic therapeutic results when combined with anti-PD-1 nivolumab treatment, providing an objective tumor response in 40% in pretreated patients with advanced colorectal or gastric cancer, and stable disease in 88% (S. Fukuoka et al., J Clin Oncol (2019) 37:(suppl; abst 2522)). Regorafenib also exhibited synergistic therapeutic results when combined with anti-PD-L1 avelumab treatment, providing a significant increase in CD8+ T-cell infiltration in 60% of advanced colorectal patients, and stable disease in 57.3% (S. Cousin et al., J Clin Oncol (2020) 38:(15_suppl; abst 4019)). This combination compares favorably with modest clinical activity of regorafenib as single agent in lung (M. S. Kies et al., https://ascopubs.org/doi/abs/10.1200/jco.2010.28.15_supp1.7585), pancreatic (S. Bozzarelli et al., Ann Oncol (2016) 27(sup 6):692P), J. S. Salmon et al., https://ascopubs.org/doi/abs/10.1200/JCO.2017.35.15_suppl.e15751), and its FDA approved use in Colorectal, Liver cancer (Hepatocellular Carcinoma), and Stomach Cancer. Regorafenib blocks multiple protein kinases involved in tumor angiogenesis (VEGFR 1-3, TIE2), oncogenesis (KIT, RET, RAF-1, BRAF), metastasis (VEGFR3, PDGFR, FGFR), and tumor immunity (CSF1R). Blocking CSF1R inhibits the production of macrophages, resulting in a reduction in the number of tumor-associated macrophages (TAM), which exert an immunosuppressive effect on CTL similar to Treg cells. Blocking angiogenesis prevents the tumor from acquiring oxygen and nutrients from the patient's vasculature. A similar drug Axitinib also blocks some of the same protein kinases involved in tumor angiogenesis (VEGFR 1-3), metastasis (VEGFR3, PDGFR) and achieves modest clinical effect as monotherapy in colorectal cancer (C. Gravalos et al., Clin Colorectal Cancer (2018) 17(2):e323-29). However, when combined with Avelumab, an anti-PD-L1, in Kidney (Renal Cell Carcinoma) it achieved breakthrough therapy designation. Another similar drug Lenvatinib also blocks some of the same protein kinases involved in tumor angiogenesis (VEGFR 1-3), metastasis (VEGFR3, PDGFR) (S. Sarcognato et al., Clinical Liver Disease, (2019) 14 (2):62-65) and achieves modest clinical effect as monotherapy in colorectal cancer (H. Shoji et al., J Clin Oncol (2019) 37(15): 3538-3538). However, when combined with Pembrolizumab, an anti-PD-1, in Advanced Gastric Cancer it achieved 69% objective response (A. Kawazoe et al., Lancet Oncol (2020) Jun. 23, 2020, online first, https://doi.org/10.1016/S1470-2045(20)30271-0).

The data from SCT tumors shows that CD4 expression is highly correlated with expression of many of the above targets. Tables 6 and 7 show the correlation between CD4 and CD8 expression in CRC tumors with many of the regorafenib targets.

TABLE 6
Pearson Correlations (%) of Expression with CD4/CD8A in SCT tumors

| | | | VEGFR1 | VEGFR2 | VEGFR3 | |
|---|---|---|---|---|---|---|
| SCT | % | CSF1R | (FLT1) | (KDR) | (FLT4) | TIE2 |
| Colorectal | CD4 | 92 | 58 | 62 | 66 | 66 |
| (N = 367) | CD8A | 49 | 23 | 23 | 30 | 26 |
| Pancreatic | CD4 | 89 | 36 | 49 | 46 | 59 |
| (N = 179) | CD8A | 58 | 17 | 43 | 44 | 66 |
| Prostate | CD4 | 85 | 17 | 31 | 31 | 46 |
| (N = 492) | CD8A | 54 | 4 | 11 | 15 | 27 |
| Head and Neck | CD4 | 91 | 39 | 49 | 40 | 54 |
| (N = 519) | CD8A | 49 | 4 | 14 | 14 | 15 |
| Melanoma | CD4 | 83 | 15 | 15 | 27 | 20 |
| (N = 471) | CD8A | 50 | 1 | −6 | 3 | 3 |
| Lung | CD4 | 90 | 38 | 24 | 45 | 50 |
| (N = 969) | CD8A | 36 | 6 | 1 | 15 | 16 |
| Esophageal | CD4 | 82 | 29 | 31 | 42 | 38 |
| (N = 182) | CD8A | 38 | 3 | 2 | 11 | 11 |
| Liver | CD4 | 78 | 28 | 25 | 26 | 36 |
| (N = 369) | CD8A | 55 | 10 | 3 | 6 | 10 |
| Stomach | CD4 | 82 | 26 | 30 | 34 | 36 |
| (N = 408) | CD8A | 45 | 16 | 13 | 21 | 18 |
| Breast | CD4 | 80 | 14 | 21 | 17 | 26 |
| (N = 1085) | CD8A | 31 | −1 | 8 | 7 | 15 |

TABLE 7
Pearson Correlations (%) of Expression with CD4/CD8A in SCT tumors

| | | | | | PD-1 | PD-L1 |
|---|---|---|---|---|---|---|
| SCT | % | FGFR1 | PDGFR-A | PDGFR-B | (PDCD1) | (CD274) |
| Colorectal | CD4 | 66 | 62 | 68 | 65 | 35 |
| (N = 367) | CD8A | 26 | 16 | 24 | 86 | 50 |
| Pancreatic | CD4 | 34 | 60 | 52 | 57 | 55 |
| (N = 179) | CD8A | 34 | 46 | 29 | 74 | 37 |
| Prostate | CD4 | 33 | 27 | 55 | 71 | 67 |
| (N = 492) | CD8A | 17 | 9 | 24 | 83 | 56 |
| Head and Neck | CD4 | 28 | 39 | 43 | 67 | 25 |
| (N = 519) | CD8A | 28 | 9 | 1 | 88 | 33 |
| Melanoma | CD4 | −3 | 34 | 33 | 67 | 19 |
| (N = 471) | CD8A | −11 | 9 | 4 | 93 | 32 |
| Lung | CD4 | 0 | 16 | 47 | 49 | 18 |
| (N = 969) | CD8A | −4 | 4 | 6 | 69 | 31 |
| Esophageal | CD4 | 2 | 30 | 44 | 73 | 13 |
| (N = 182) | CD8A | −4 | 4 | 9 | 87 | 28 |
| Liver | CD4 | 34 | 22 | 37 | 23 | 37 |
| (N = 369) | CD8A | 34 | 21 | 19 | 50 | 54 |
| Stomach | CD4 | 11 | 26 | 42 | 54 | 20 |
| (N = 408) | CD8A | 4 | 2 | 16 | 60 | 35 |
| Breast | CD4 | −8 | 7 | 21 | 60 | 49 |
| (N = 1085) | CD8A | −7 | 3 | 0 | 81 | 49 |

The correlations between T-cell markers (CD4 and CD8A) and the listed target genes indicate stronger stimulation of the tumor defenses for tumor angiogenesis, oncogenesis, and metastasis, and tumor immunity from T-cells in colorectal, pancreatic, head and neck, and lung cancer (M. Kissel et al., Oncotarget (2017) 8(63):107096-108). These correlations indicate that regorafenib was synergistic with nivolumab, suggesting it will also be synergistic with a T-cell activating vaccine.

The data from solid cancer tumors also shows that for each cancer, survival is closely correlated with expression of the proteins corresponding to one of the above target receptors. Table 8 shows the COX Proportional Hazards Ratio for each cancer that compares the overall survival of patients in the top quartile of expression of the protein that binds to a regorafenib target with a strong relationship to survival in the bottom quartile. VEGF-B binds to VEGFR1, VEGFA binds to VEGFR1 and VEGFR2, and VEGFC binds to VEGFR3 (https://www.ncbi.nlm.nih.gov/pmc/articles/PMC3411125/). These hazard ratios in Table 8 indicate that regorafenib will have beneficial activity against angiogenesis in cancers beyond colorectal and gastric, and together with the correlations in Table 8 suggest they will also be synergistic with a T-cell activating vaccine. The findings presented here are consistent with the results reported by S. Zong et al., Clin Chim Acta (2016) 458:106-14 (CRC); Z. Zhang et al., PLoS ONE (2016) 11(11):e0165725 (breast); H. Xia et al., Cancer Biomark (2016) 17(2):165-70 (esophageal); W. Cao et al., Tumour Biol (2014) 35(4):3377-83 (gastric); and H. Jiang et al., Clin Chim Acta (2014) 427:94-99 (non-small cell lung cancer).

TABLE 8
COX Proportional Hazards Ratio closely
correlated targets in SCT Tumors

| | | | Cox |
|---|---|---|---|
| Solid | Number | Protein that binds to target | Proportional |
| Cancerous | of patient | receptor that has a strong | Hazards Ratio |
| Tumor (SCT) | tumors | relationship to survival | (%) |
| Colorectal | 367 | VEGFC | 190 |
| Pancreatic | 179 | VEGFA | 130 |
| Prostate | 492 | VEGFA | 250 |
| Head and Neck | 519 | VEGFC | 180 |
| Melanoma | 471 | VEGFB | 160 |
| Lung | 969 | VEGFC | 160 |
| Esophageal | 182 | VEGFC | 160% |
| Liver | 369 | VEGFC | 190 |
| Stomach | 384 | VEGFB | 200 |
| Breast | 1085 | VEGFA | 150 |

### Example 6: Bevacizumab Treatment

Tumor cells can upregulate VEGFR1 and VEGFR2 which reduces T-cell infiltration due to vascular and endothelial dysregulation. For example in Renal Cell Carcinoma patients, anti-VEGF treatment with Bevacizumab augmented intra-tumoral T-cell infiltration, potentially through vascular normalization and endothelial cell activation (Wallin et al., Nat Commun 2016 Aug. 30; 7:12624).

Similar to Regorafenib, recent clinical trial results found that treatment the monoclonal antibody bevacizumab and chemotherapy capecitabine exhibited synergistic therapeutic results when combined with anti PD-L1 atezolizumab treatment, providing an objective tumor response in 8.5% in patients with pretreated metastatic colorectal cancer with 8.5% ongoing responses at time of publication (N. Mettu et al., Annals of Oncology (2019) 30 (suppl_5): v198-v252.). This triple combination in pretreated patients compares favorably with more modest 4.4% objective response rate in the same study of bevacizumab and capecitabine in pretreated metastatic colorectal cancer patients with 2.2% ongoing responses. Bevacizumab blocks multiple protein kinases involved in tumor angiogenesis (VEGFR 1-2) by binding to circulating VEGF-A (Pandey et. al. Hypertension. 2018; 71:e1-e8). Blocking angiogenesis prevents the tumor from acquiring oxygen and nutrients from the patient's vasculature.

The data from colorectal cancer tumors in Table 6 above shows that CD4 and CD8A are closely correlated with expression of the above target receptors of VEGF-A, namely VEGFR1 and VEGFR2 in colorectal cancer. The clinical data from Example 4 showed that on removing bevacizumab from capecitabine, bevacizumab, aspirin, celecoxib and Losartan maintenance treatment, CEA and scans showed signs of progression. In contrast, when antigen presenting cell agents (neulasta and Maraviroc) and anti PD-1 were added to Capecitabine, Bevacizumab, aspirin, celecoxib and Losartan, PET scans and CEA showed a dramatic reduction in tumor burden.

### Example 7: Cetuximab Treatment

Similar to Regorafenib, recent clinical trial results found that treatment the monoclonal antibody cetuximab and chemotherapy FOLFOX exhibited synergistic therapeutic results when combined with anti PD-L1 avelumab treatment, providing an overall response rate of 80% in subjects with untreated metastatic colorectal with RAS wild type tumors (jnccn360. org/colorectal/news/avelumab-plus-folfox-and-cetuximab-in-metastatic-colorectal-cancer/). This triple combination compares favorably with more modest 66% objective response rate expected with cetuximab and FOLFOX in metastatic colorectal cancer in a similar setting (www.ncbi.nlm.nih.gov/pmc/articles/PMC6324088/). Cetuximab acts on the EGFR receptor and creates immunogenic cell death attracting dendritic cells and t-cells to the tumor (www.ncbi.nlm.nih.gov/pubmed/27135741; www.ncbi.nlm.nih.gov/pmc/articles/PMC5378263/) both in patients and pre-clinical models.

The data from colorectal cancer tumors shows that EGFR expression has a low correlation with specific markers of T-cell infiltration including CD8A, IFNG (interferon gamma, Kosmidis et al J Cancer. 2018; 9(2): 232-238 www.ncbi.nlm.nih.gov/pmc/articles/PMC5771329/) which correlates 83% with CD8A and its supporting antigen presentation machinery (Ozcan et al Oncoimmunology. 2018; 7(7): e1445453, www.ncbi.nlm.nih.gov/pmc/articles/PMC5993484/) described by TAP1 and B2M which both correlate 56% with CD8A. This indicates that some patients with low T-cell infiltration, low levels of interferon gamma, and dysfunctional antigen presentation machinery express EGFR and can benefit from Cetuximab treatment.

TABLE 9

| SCT | % | IFNG | TAP1 | B2M | CD8A |
|---|---|---|---|---|---|
| Colorectal | EGFR | −3% | 8% | 15% | 6% |
| (N = 367) | IFNG | 100% | 66% | 48% | 83% |
| | TAP1 | — | 100% | 55% | 56% |
| | B2M | — | — | 100% | 56% |
| | CD8A | — | — | — | 100% |

### EMBODIMENTS

An embodiment of the invention is a system for treating a solid cancerous tumor (SCT) in a subject, the system having: an antigen presenting cell agent; a T-cell activating vaccine; and an immunosuppression inhibitor. An wherein the antigen presenting cell agent is a CD40 agonist, a Toll-like receptor agonist, an adjuvant, FLT3L, or any combination thereof. An embodiment is the system wherein the immunosuppression inhibitor is selected from the group consisting of a CD73 inhibitor, a PD-L1 inhibitor, a PD-1 inhibitor, an A2a receptor inhibitor, a multi-kinase inhibitor, cyclophosphamide, a COX-2 inhibitor, a prostaglandin-E2 inhibitor, and any combination thereof. An embodiment is the system further having an angiotensin II type 1 receptor antagonist. An embodiment is the system wherein the immunosuppression inhibitor is a PD-1 inhibitor, a PD-L1 inhibitor, or a multi-kinase inhibitor. An embodiment is the system wherein the immunosuppression inhibitor is a PD-1 inhibitor, a PD-L1 inhibitor, or a multi-kinase inhibitor. An embodiment is the system wherein the immunosuppression inhibitor is a CD73 inhibitor and a PD-L1 inhibitor. An embodiment is the system wherein the immunosuppression inhibitor is a COX-2 inhibitor, a multi-kinase inhibitor, and a PD-1 inhibitor or PD-L1 inhibitor. An embodiment is the system wherein the multi-kinase inhibitor is regorafenib, sorafenib, fruquintinib, axitinib, or lenvatinib. An embodiment is the system wherein the PD-1 inhibitor is nivolumab. An embodiment is the system wherein the PD-L1 inhibitor is durvalumab.

An embodiment is the system which further comprises radiation therapy. An embodiment is the system wherein the radiation therapy is stereotactic body radiation therapy (SBRT). An embodiment is the system wherein the SCT is selected from colorectal cancer, pancreatic cancer, prostate cancer, head and neck cancer, lung cancer, melanoma, breast cancer, liver cancer, esophageal cancer, and gastric cancer.

An embodiment is the system wherein the T-cell activating vaccine comprises a neoantigen vaccine. An embodiment is the system wherein the neoantigen vaccine comprises a plurality of neoantigen peptides or multi-antigen polypeptides, or nucleic acids encoding a plurality of neoantigen peptides or multi-antigen polypeptides. An embodiment is the system wherein the plurality of neoantigen peptides or multi-antigen polypeptides consists of about 3 to about 50 polypeptides. An embodiment is the system wherein the plurality of neoantigen peptides or multi-antigen polypeptides consists of about 5 to about 40 polypeptides. An embodiment is the system wherein the plurality of neoantigen peptides or multi-antigen polypeptides consists of about 10 to about 30 polypeptides. An embodiment is the system wherein the plurality of neoantigen peptides or multi-antigen polypeptides comprises one or more short neoantigen polypeptides. An embodiment is the system wherein the short neoantigens are about 6 to about 12 amino acids in length. An embodiment is the system wherein the short neoantigens are about 8 to about 10 amino acids in length. An embodiment is the system wherein the plurality of neoantigen peptides or multi-antigen polypeptides comprises one or more long neoantigens. An embodiment is the system wherein the long neoantigens are about 12 to about 30 amino acids in length. An embodiment is the system wherein the long neoantigens are about 15 to about 24 amino acids in length.

An embodiment is the system wherein the neoantigen peptides or multi-antigen polypeptides are designed to correspond to antigens expressed by the subject's SCT. An embodiment is the system wherein the plurality of neoantigen peptides or multi-antigen polypeptides is provided as a plurality of subgroups, wherein each subgroup comprises at least one neoantigen that is not present in at least one other subgroup. An embodiment is the system wherein the plurality of subgroups consists of about two to about ten subgroups. An embodiment is the system wherein the plurality of subgroups consists of about three to about eight subgroups. An embodiment is the system wherein each subgroup comprises about three to about 20 neoantigens. An embodiment is the system wherein each subgroup comprises about five to about ten neoantigens.

An embodiment is the system wherein the antigen presenting cell agent is FLT3L; and the immunosuppression inhibitor is selected from a CD73 inhibitor, a multi-kinase inhibitor, a PD-1 inhibitor, a PD-L1 inhibitor, aspirin, and celecoxib, or a combination thereof. An embodiment is the system wherein the antigen presenting cell agent comprises FLT3L; and the immunosuppression inhibitor comprises regorafenib, nivolumab, and aspirin.

An aspect of the invention is a T-cell activating vaccine for treating SCT in a subject, having a plurality of neoantigens, or one or more nucleic acids encoding a plurality of neoantigens, wherein the plurality of neoantigens comprises at least one short neoantigen. An embodiment is the T-cell activating vaccine having at least one long neoantigen; and a pharmaceutically acceptable carrier. An embodiment is the vaccine wherein the plurality of neoantigens consists of about 3 to about 50 neoantigens. An embodiment is the vaccine wherein the plurality of neoantigen peptides and/or multi-antigen polypeptides comprises about 5 to about 40 neoantigens. An embodiment is the vaccine wherein the plurality of neoantigens comprises about 10 to about 30 neoantigen peptides and/or multi-antigen polypeptides. An embodiment is the vaccine wherein the short neoantigens are about 6 to about 12 amino acids in length. An embodiment is the vaccine wherein the short neoantigens are about 8 to about 10 amino acids in length. An embodiment is the vaccine wherein the plurality of neoantigens comprises one or more long neoantigens. An embodiment is the vaccine wherein the long neoantigens are about 12 to about 30 amino acids in length. An embodiment is the vaccine wherein the long neoantigens are about 15 to about 24 amino acids in length. An embodiment is the vaccine wherein the neoantigen peptides or multi-antigen polypeptides are designed to correspond to antigens expressed by the subject's SCT.

An embodiment is the vaccine wherein the plurality of neoantigens is provided as a plurality of subgroups, wherein each subgroup comprises at least one neoantigen that is not present in at least one other subgroup. An embodiment is the vaccine wherein the plurality of subgroups consists of about two to about ten subgroups. An embodiment is the vaccine wherein the plurality of subgroups consists of about three to about eight subgroups. An embodiment is the vaccine wherein each subgroup comprises about three to about 20 neoantigens. An embodiment is the vaccine wherein each subgroup comprises about five to about ten neoantigens. An embodiment is the vaccine further comprising an adjuvant. An embodiment is the vaccine further comprising an antigen presenting cell agent.

An aspect of the invention is the method for treating SCT in a subject, wherein the method includes a) administering an effective amount of an antigen presenting cell agent selected from the group consisting of: a CD40 agonist, a Toll-like receptor agonist, an adjuvant, FLT3L, and any combination thereof; b) administering an effective amount of a T-cell activating vaccine; and c) administering an effective amount of an immunosuppression inhibitor selected from the group consisting of a CD73 inhibitor, a PD-1 inhibitor, a PD-L1 inhibitor, an A2a receptor inhibitor, a multi-kinase inhibitor, cyclophosphamide, a COX-2 inhibitor, a prostaglandin-E2 inhibitor, and any combination thereof. An embodiment is the method further including d) administering an effective amount of an angiotensin II type 1 receptor antagonist. An embodiment is the method wherein the SCT is selected from colorectal cancer, pancreatic cancer, prostate cancer, head and neck cancer, lung cancer, melanoma, breast cancer, liver cancer, esophageal cancer, and gastric cancer

An embodiment is the method wherein the antigen presenting cell agent is administered prior to administering the T-cell activating vaccine and the immunosuppression inhibitor. An embodiment is the method wherein at least one antigen presenting cell agent is administered for about 1 day to about 30 days prior to administering a T-cell activating vaccine. An embodiment is the method wherein the activation state, population size, or distribution of antigen presenting cells is measured prior to administering a T-cell activating vaccine. An embodiment is the method wherein the T-cell activating vaccine is administered only after the activation state, population size, or distribution of antigen presenting cells reaches a pre-determined value. An embodiment is the method wherein the Toll-like receptor agonist is poly(I:C) or poly-ICLC. An embodiment is the method wherein the adjuvant is MONTANIDE™ or DepoVax™. An embodiment is the method wherein the COX-2 inhibitor is aspirin. An embodiment is the method wherein the COX-2 inhibitor is ibuprofen. An embodiment is the method wherein the COX-2 inhibitor is naproxen. An embodiment is the method wherein the COX-2 inhibitor is indomethacin. An embodiment is the method wherein the COX-2 inhibitor is celecoxib.

An embodiment is the method wherein step a) includes administering an effective amount of FLT3L, poly-ICLC, or a CD40 agonist, or a combination thereof; and administering an effective amount of a COX-2 inhibitor. An embodiment is the method wherein each agent is administered independently. An embodiment is the method wherein two or more antigen presenting cell agents are combined in a single formulation.

An embodiment is the method wherein the T-cell activating vaccine comprises a neoantigen vaccine. An embodiment is the method wherein the neoantigen vaccine comprises a plurality of neoantigen peptides or multi-antigen polypeptides, or one or more nucleic acids encoding a plurality of neoantigen peptides or multi-antigen polypeptides. An embodiment is the method wherein the plurality of neoantigens consists of about 3 to about 50 neoantigens. An embodiment is the method wherein the plurality of neoantigens consists of about 5 to about 40 neoantigens. An embodiment is the method wherein the plurality of neoantigens consists of about 10 to about 30 polypeptides.

An embodiment is the method wherein the plurality of neoantigens is administered by injection at a plurality of injection sites. An embodiment is the method wherein the plurality of injection sites is selected to deliver neoantigens to different lymph nodes. An embodiment is the method wherein the plurality of injection sites comprises about two to about ten different injection sites. An embodiment is the method wherein the plurality of injection sites comprises about three to about seven different injection sites. An embodiment is the method wherein a subset of the plurality of neoantigens is administered at each injection site. An embodiment is the method wherein the subset of the plurality of neoantigens contains about two to about seven neoantigens. An embodiment is the method wherein the subset of the plurality of neoantigens contains about five neoantigens.

An embodiment is the method wherein the subsets of the plurality of neoantigens together comprise the T-cell activating vaccine, and wherein at least one of the subsets contain at least two neoantigens that are not present in at least one of the other subsets. An embodiment is the method wherein the neoantigens are designed to correspond to antigens expressed by the subject's SCT and are not expressed by normal tissue.

An embodiment is the method further including identifying one or more neoantigens expressed in the subject's SCT, and preparing the T-cell activating vaccine using a neoantigen peptide and/or multi-antigen polypeptide that corresponds to a neoantigen expressed in the subject's SCT. An embodiment is the method wherein a neoantigen expressed in the subject's SCT is identified by whole exome sequencing. An embodiment is the method wherein the plurality of neoantigens comprises one or more short neoantigens. An embodiment is the method wherein the short neoantigens are about 6 to about 12 amino acids in length. An embodiment is the method wherein the short neoantigens are about 8 to about 10 amino acids in length. An embodiment is the method wherein the plurality of neoantigens comprises one or more long neoantigens. An embodiment is the method wherein the long neoantigens are about 12 to about 30 amino acids in length. An embodiment is the method wherein the long neoantigens are about 15 to about 24 amino acids in length.

An embodiment is the method wherein the administration of the T-cell activating vaccine is repeated one, two, or three times. An embodiment is the method wherein the T-cell activating vaccine is administered with an adjuvant or a Toll-like receptor agonist. An embodiment is the method wherein the activation state, population size, or distribution of T-cells is determined after administering the T-cell activating vaccine. An embodiment is the method wherein the activation state, population size, or distribution of T-cells is determined about 5 days to about 30 days after administering the T-cell activating vaccine. An embodiment is the method wherein the T-cell activating vaccine is administered again if the activation state, population size, or distribution of T-cells has not reached a pre-determined value. An embodiment is the method wherein the T-cell activating vaccine is administered a third time if the activation state, population size, or distribution of T-cells has not reached a pre-determined value.

An embodiment is the method wherein a second T-cell activating vaccine is administered if the activation state, population size, or distribution of T-cells has not reached a pre-determined value, wherein the second T-cell activating vaccine comprises at least one antigen that was not present in the first T-cell activating vaccine. An embodiment is the method wherein the immunosuppression inhibitor is administered about 1 days to about 30 days after the last administration of the T-cell activating vaccine. An embodiment is the method wherein step c) comprises administering a CD73 inhibitor and a PD-L1 inhibitor. An embodiment is the method wherein the immunosuppression inhibitor comprises a multi-kinase inhibitor, a PD-1 inhibitor, or a PD-L1 inhibitor. An embodiment is the method wherein the immunosuppression inhibitor comprises multi-kinase inhibitor and a PD-1 inhibitor. An embodiment is the method wherein the multi-kinase inhibitor comprises regorafenib, sorafenib, fruquintinib, axitinib, or lenvatinib. An embodiment is the method wherein the PD-1 inhibitor comprises nivolumab. An embodiment is the method wherein the PD-L1 inhibitor comprises durvalumab.

An embodiment is the method wherein the prostaglandin E2 inhibitor is a PTGES2 inhibitor. An embodiment is the method wherein the angiotensin II type 1 receptor antagonist comprises losartan or a pharmaceutically acceptable salt thereof. An embodiment is the method wherein administration of an antigen presenting cell agent is continued for about 1 day to about 30 days. An embodiment is the method wherein administration of a T-cell activating vaccine is continued for about 1 day to about 60 days. An embodiment is the method wherein administration of an immunosuppression inhibitor is continued for about 1 day to about 90 days. An embodiment is the method wherein the SCT response to treatment is measured. An embodiment is the method wherein administration of an antigen presenting cell agent is continued until the SCT response to treatment reaches a pre-determined value. An embodiment is the method wherein administration of a T-cell activating vaccine is continued until the SCT response to treatment reaches a pre-determined value. An embodiment is the method wherein administration of an immunosuppression inhibitor is continued until the SCT response to treatment reaches a pre-determined value.

An embodiment is the method which further includes irradiating the SCT. An embodiment is the method wherein the irradiation is stereotactic body radiation therapy (SBRT). An embodiment is the method wherein the SCT comprises metastatic CRC (mCRC). An embodiment is the method wherein the cancer comprises microsatellite stable mCRC (MSS mCRC). An embodiment is the method wherein steps (a), (b), and (c) are performed in the order (b), (a), (c); (b), (c), (a), (c); (a), (b), (c); or (a), (b), (a), (c). An embodiment is the method wherein two or more of steps (a), (b), and (c) are performed simultaneously.

What is claimed is:

1. A system for treating a solid cancerous tumor (SCT) in a subject, the system comprising:
(a) an antigen presenting cell agent;
(b) a T-cell activating vaccine; and
(c) an immunosuppression inhibitor.

2. The system of claim 1, wherein the antigen presenting cell agent is selected from the group consisting of a CD40 agonist, a Toll-like receptor agonist, an adjuvant, FLT3L, and any combination thereof.

3. The system of claim 1 or 2, wherein the immunosuppression inhibitor is selected from the group consisting of a CD73 inhibitor, a PD-L1 inhibitor, a PD-1 inhibitor, an A2a receptor inhibitor, a multi-kinase inhibitor, cyclophosphamide, a COX-2 inhibitor, a prostaglandin-E2 inhibitor, and any combination thereof.

4. The system of any one of claims 1 to 3, further comprising (d) an angiotensin II type 1 receptor antagonist.

5. The system of any one of claims 1 to 4, wherein the immunosuppression inhibitor comprises a PD-1 inhibitor, a PD-L1 inhibitor, or a multi-kinase inhibitor.

6. The system of any one of claims 1 to 5, wherein the multi-kinase inhibitor comprises regorafenib, sorafenib, fruquintinib, axitinib, or lenvatinib.

7. The system of any one of claims 1 to 6, wherein the PD-1 inhibitor comprises nivolumab.

8. The system of any one of claims 1 to 7, wherein the PD-L1 inhibitor comprises durvalumab.

9. The system of any one of claims 1 to 8, further comprising (e) therapeutic radiation.

10. The system of any one of claims 1 to 9, wherein the SCT is selected from colorectal cancer, pancreatic cancer, prostate cancer, head and neck cancer, lung cancer, melanoma, breast cancer, liver cancer, esophageal cancer, and gastric cancer.

11. The system of any one of claims 1 to 10, wherein the T-cell activating vaccine comprises a neoantigen vaccine.

12. The system of claim 11, wherein the neoantigen vaccine comprises a plurality of neoantigen peptides or multi-antigen polypeptides, or nucleic acids encoding a plurality of neoantigen peptides or multi-antigen polypeptides.

13. The system of claim 12, wherein the plurality of neoantigen peptides or multi-antigen polypeptides consists of about 3 to about 50 neoantigens.

14. The system of claim 13, wherein the plurality of neoantigen peptides or multi-antigen polypeptides consists of about 5 to about 40 neoantigens.

15. The system of claim 14, wherein the plurality of neoantigen peptides or multi-antigen polypeptides consists of about 10 to about 30 neoantigens.

16. The system of any one of claims 11 to 15, wherein the plurality of neoantigen peptides or multi-antigen polypeptides comprises one or more short neoantigens.

17. The system of claim 16, wherein the short neoantigens are about 6 to about 12 amino acids in length.

18. The system of claim 17, wherein the short neoantigens are about 8 to about 10 amino acids in length.

19. The system of any one of claims 11 to 18, wherein the plurality of neoantigen peptides or multi-antigen polypeptides comprises one or more long neoantigens.

20. The system of claim 19, wherein the long neoantigens are about 12 to about 30 amino acids in length.

21. The system of claim 20, wherein the long neoantigens are about 15 to about 24 amino acids in length.

22. The system of any one of claims 11 to 21, wherein the neoantigen peptides or multi-antigen polypeptides are designed to correspond to antigens expressed by the subject's SCT, and not by normal tissue.

23. The system of any one of claims 12 to 22, wherein the plurality of neoantigen peptides or multi-antigen polypeptides is provided as a plurality of subgroups, wherein each subgroup comprises at least one neoantigen that is not present in at least one other subgroup.

24. The system of claim 23, wherein the plurality of subgroups consists of about two to about ten subgroups.

25. The system of claim 24, wherein the plurality of subgroups consists of about three to about eight subgroups.

26. The system of any one of claims 23 to 25, wherein each subgroup comprises about three to about 20 neoantigens.

27. The system of claim 26, wherein each subgroup comprises about five to about ten neoantigens.

28. The system of any one of claims 1 to 27, wherein:
the antigen presenting cell agent is FLT3L; and
the immunosuppression inhibitor is selected from a COX-2 inhibitor, a prostaglandin-E2 inhibitor, a CD73 inhibitor, a multi-kinase inhibitor, a PD-1 inhibitor, and a PD-L1 inhibitor, or a combination thereof.

29. The system of claim 28, wherein
the antigen presenting cell agent comprises FLT3L; and
the immunosuppression inhibitor comprises regorafenib, nivolumab, and aspirin.

30. A T-cell activating vaccine for treating an SCT in a subject, comprising:
a plurality of neoantigen peptides or multi-antigen polypeptides, or one or more nucleic acids encoding a plurality of neoantigen peptides or multi-antigen polypeptides, wherein the plurality of neoantigen peptides or multi-antigen polypeptides comprises at least one short neoantigen; and
a pharmaceutically acceptable carrier.

31. The T-cell activating vaccine of claim 30, wherein the plurality of neoantigen peptides or multi-antigen polypeptides comprises at least one long neoantigen.

32. The T-cell activating vaccine of claim 30 or 31, wherein the plurality of neoantigen peptides or multi-antigen polypeptides comprises about 3 to about 50 neoantigens.

33. The T-cell activating vaccine of claim 32, wherein the plurality of neoantigen peptides or multi-antigen polypeptides comprises about 5 to about 40 neoantigens.

34. The T-cell activating vaccine of claim 33, wherein the plurality of neoantigen polypeptides consists of about 10 to about 30 polypeptides.

35. The T-cell activating vaccine of any one of claims 30 to 34, wherein the short neoantigens are about 6 to about 12 amino acids in length.

36. The T-cell activating vaccine of claim 35, wherein the short neoantigens are about 8 to about 10 amino acids in length.

37. The T-cell activating vaccine of any one of claims 30 to 36, wherein the plurality of neoantigen peptides or multi-antigen polypeptides comprises one or more long neoantigens.

38. The T-cell activating vaccine of claim 37, wherein the long neoantigens are about 12 to about 30 amino acids in length.

39. The T-cell activating vaccine of claim 38, wherein the long neoantigens are about 15 to about 24 amino acids in length.

40. The T-cell activating vaccine of any one of claims 30 to 39, wherein the neoantigen peptides or multi-antigen polypeptides are designed to correspond to neoantigens expressed by the subject's SCT.

41. The T-cell activating vaccine of any one of claims 30 to 40, wherein the plurality of neoantigen peptides or multi-antigen polypeptides is provided as a plurality of subgroups, wherein each subgroup comprises at least one neoantigen that is not present in at least one other subgroup.

42. The T-cell activating vaccine of claim 41, wherein the plurality of subgroups consists of about two to about ten subgroups.

43. The T-cell activating vaccine of claim 42, wherein the plurality of subgroups consists of about three to about eight subgroups.

44. The T-cell activating vaccine of any one of claims 41 to 43, wherein each subgroup comprises about three to about 20 neoantigen peptides or multi-antigen polypeptides.

45. The T-cell activating vaccine of claim 44, wherein each subgroup comprises about five to about ten neoantigen peptides or multi-antigen polypeptides.

46. The T-cell activating vaccine of any one of claims 30 to 45, further comprising an adjuvant.

47. The T-cell activating vaccine of any one of claims 30 to 45, further comprising an antigen presenting cell agent.

48. A method for treating an SCT in a subject, the method comprising:
(a) administering an effective amount of an antigen presenting cell agent selected from the group consisting of: a CD40 agonist, a Toll-like receptor agonist, an adjuvant, FLT3L, and any combination thereof;
(b) administering an effective amount of a T-cell activating vaccine; and
(c) administering an effective amount of an immunosuppression inhibitor selected from the group consisting of a CD73 inhibitor, a PD-1 inhibitor, a PD-L1 inhibitor, an A2a receptor inhibitor, a multi-kinase inhibitor, cyclophosphamide, a COX-2 inhibitor, a prostaglandin-E2 inhibitor, and any combination thereof.

49. The method of claim 48, further comprising: (d) administering an effective amount of an angiotensin II type 1 receptor antagonist.

50. The method of claim 48 or 49, wherein the SCT is selected from colorectal cancer, pancreatic cancer, prostate cancer, head and neck cancer, lung cancer, melanoma, breast cancer, liver cancer, esophageal cancer, and gastric cancer

51. The method of any one of claims 48 to 50, wherein the antigen presenting cell agent is administered prior to administering the T-cell activating vaccine and the immunosuppression inhibitor.

52. The method of claim 51, wherein at least one antigen presenting cell agent is administered for about 1 day to about 30 days prior to administering a T-cell activating vaccine.

53. The method of claim 51, wherein the activation state, population size, or distribution of antigen presenting cells is measured prior to administering a T-cell activating vaccine.

54. The method of claim 53, wherein the T-cell activating vaccine is administered only after the activation state, population size, or distribution of antigen presenting cells reaches a pre-determined value.

55. The method of any one of claims 48 to 54, wherein the Toll-like receptor agonist comprises poly(I:C) or poly-ICLC.

56. The method of any one of claims 48 to 55, wherein the adjuvant comprises MONTANIDE™ or DepoVax™.

57. The method of any one of claims 48 to 56, wherein the COX-2 inhibitor comprises aspirin.

58. The method of any one of claims 48 to 57, wherein step a) comprises:
administering an effective amount of FLT3L, poly-ICLC, or a CD40 agonist, or a combination thereof; and
administering an effective amount of a COX-2 inhibitor.

59. The method of claim 58, wherein each agent is administered independently.

60. The method of any one of claims 48 to 58, wherein two or more antigen presenting cell agents are combined in a single formulation.

61. The method of any one of claims 48 to 59, wherein the T-cell activating vaccine comprises a neoantigen vaccine.

62. The method of claim 61, wherein the neoantigen vaccine comprises a plurality of neoantigen peptides or multi-antigen polypeptides, or one or more nucleic acids encoding a plurality of neoantigen peptides or multi-antigen polypeptides.

63. The method of claim 62, wherein the plurality of neoantigen peptides or multi-antigen polypeptides comprises about 3 to about 50 neoantigens.

64. The method of claim 63, wherein the plurality of neoantigen polypeptides comprises about 5 to about 40 neoantigens.

65. The method of claim 64, wherein the plurality of neoantigen polypeptides comprises about 10 to about 30 neoantigens.

66. The method of any one of claims 62 to 65, wherein the plurality of neoantigen peptides or multi-antigen polypeptides is administered by injection at a plurality of injection sites.

67. The method of claim 66, wherein the plurality of injection sites is selected to deliver neoantigen peptides or multi-antigen polypeptides to different lymph nodes.

68. The method of claim 66 or 67, wherein the plurality of injection sites comprises about two to about ten different injection sites.

69. The method of claim 68, wherein the plurality of injection sites comprises about three to about seven different injection sites.

70. The method of any one of claims 66 to 69, wherein a subset of the plurality of neoantigen polypeptides is administered at each injection site.

71. The method of claim 70, wherein the subset of the plurality of neoantigen peptides or multi-antigen polypeptides comprises about two to about seven neoantigen peptides or multi-antigen polypeptides.

72. The method of claim 71, wherein the subset of the plurality of neoantigen peptides or multi-antigen polypeptides contains about five neoantigens.

73. The method of any one of claims 62 to 72, wherein the subsets of the plurality of neoantigen peptides or multi-antigen polypeptides together comprise the T-cell activating vaccine, and wherein at least one of the subsets contain at least two neoantigen polypeptides that are not present in at least one of the other subsets.

74. The method of any one of claims 61 to 73, wherein the neoantigen peptides or multi-antigen polypeptides are designed to correspond to neoantigens expressed by the subject's SCT.

75. The method of any one of claims 61 to 73, further comprising (e) identifying one or more neoantigens expressed in the subject's SCT, and preparing the T-cell activating vaccine using a neoantigen peptide or multi-antigen polypeptide that corresponds to a neoantigen expressed in the subject's SCT and not in normal tissue.

76. The method of claim 75, wherein a neoantigen expressed in the subject's SCT is identified by whole exome sequencing.

77. The method of any one of claims 62 to 76, wherein the plurality of neoantigen peptides or multi-antigen polypeptides comprises one or more short neoantigens.

78. The method of claim 77, wherein the short neoantigens are about 6 to about 12 amino acids in length.

79. The method of claim 78, wherein the short neoantigens are about 8 to about 10 amino acids in length.

80. The method of any one of claims 62 to 79, wherein the plurality of neoantigen peptides or multi-antigen polypeptides comprises one or more long neoantigens.

81. The method of claim 80, wherein the long neoantigens are about 12 to about 30 amino acids in length.

82. The method of claim 81, wherein the long neoantigens are about 15 to about 24 amino acids in length.

83. The method of any one of claims 48 to 82, wherein the administration of the T-cell activating vaccine is repeated one, two, or three times.

84. The method of any one of claims 48 to 83, wherein the T-cell activating vaccine is administered with an adjuvant or a Toll-like receptor agonist.

85. The method of any one of claims 48 to 84, wherein the activation state, population size, or distribution of T-cells is determined after administering the T-cell activating vaccine.

86. The method of claim 85, wherein the activation state, population size, or distribution of T-cells is determined about 5 days to about 30 days after administering the T-cell activating vaccine.

87. The method of claim 85 or 86, wherein the T-cell activating vaccine is administered again if the activation state, population size, or distribution of T-cells has not reached a pre-determined value.

88. The method of any one of claims 85 to 87, wherein step (e) is repeated.

89. The method of any one of claims 85 to 88, wherein a second T-cell activating vaccine is administered if the activation state, population size, or distribution of T-cells has not reached a pre-determined value, wherein the second T-cell activating vaccine comprises at least one antigen that was not present in the first T-cell activating vaccine.

90. The method of any one of claims 48 to 89, wherein the immunosuppression inhibitor is administered about 1 days to about 30 days after the last administration of the T-cell activating vaccine.

91. The method of any one of claims 48 to 90, wherein step c) comprises administering a CD73 inhibitor and a PD-L1 inhibitor.

92. The method of any one of claims 48 to 91, wherein the immunosuppression inhibitor comprises a multi-kinase inhibitor, a PD-1 inhibitor, or a PD-L1 inhibitor.

93. The method of claim 92, wherein the immunosuppression inhibitor comprises multi-kinase inhibitor and a PD-1 inhibitor.

94. The method of claim 93, wherein the multi-kinase inhibitor comprises regorafenib, sorafenib, fruquintinib, axitinib, or lenvatinib.

95. The method of any one of claims 92 to 94, wherein the PD-1 inhibitor comprises nivolumab.

96. The method of any one of claims 92 to 94, wherein the PD-L1 inhibitor comprises durvalumab.

97. The method of any one of claims 48 to 96, wherein the prostaglandin E2 inhibitor is a PTGES2 inhibitor.

98. The method of any one of claims 49 to 97, wherein the angiotensin II type 1 receptor antagonist comprises losartan or a pharmaceutically acceptable salt thereof.

99. The method of any one of claims 48 to 98, wherein administration of an antigen presenting cell agent is continued for about 1 day to about 30 days.

100. The method of any one of claims 48 to 99, wherein administration of a T-cell activating vaccine is continued for about 1 day to about 60 days.

101. The method of any one of claims 48 to 100, wherein administration of the immunosuppression inhibitor is continued for about 1 day to about 90 days.

102. The method of any one of claims 48 to 101, wherein the SCT response to treatment is measured.

103. The method of claim 102, wherein administration of an antigen presenting cell agent is continued until the SCT response to treatment reaches a pre-determined value.

104. The method of claim 102 or 103, wherein administration of a T-cell activating vaccine is continued until the SCT response to treatment reaches a pre-determined value.

105. The method of any one of claims 102 to 104, wherein administration of the immunosuppression inhibitor is continued until the SCT response to treatment reaches a pre-determined value.

106. The method of any one of claims 48 to 105, further comprising: (f) irradiating the SCT.

107. The method of claim 106, wherein the irradiating comprises stereotactic body radiation therapy (SBRT).

108. The method of any one of claims 48 to 107, wherein the SCT comprises metastatic CRC (mCRC).

109. The method of claim 108, wherein the cancer comprises microsatellite stable mCRC (MSS mCRC).

110. The method of any one of claims 48, 49, and 55-109, wherein steps (a), (b), and (c) are performed in the order
(i) (b), (a), (c);
(ii) (b), (c), (a), (c);
(iii) (a), (b), (c); or
(iv) (a), (b), (a), (c).

111. The method of any one of claims 48, 49, and 55-110, wherein two or more of steps (a), (b), (c) and (d) are performed simultaneously.
